(19) United States
(12) Patent Application Publication
(10) Pub. No.: US 2026/0358328 A1
(43) Pub. Date: Nov. 12, 2026

(54) Air-Dried Cell Monolayers and Methods of Preparing the Same

(71) Applicant: ETH Zurich, Zurich (CH)

(72) Inventors: Yannik Severin, Zurich (CH); Berend Snijder, Zurich (CH)

(21) Appl. No.: 19/129,069

(22) PCT Filed: Nov. 15, 2023
(86) PCT No.: PCT/EP2023/081979

§ 371 Date: May 12, 2025

(30) Foreign Application Priority Data
Nov. 15, 2022 (EP) 22207433.8

(51) Int. Cl. G01N 33/50; C12M 1/00; C12M 1/32; C12N 5/09; G01N 1/30

(52) U.S. Cl. CPC G01N 33/5011; C12M 23/12; C12M 47/14; C12N 5/0693; G01N 1/30

(57) ABSTRACT

The invention relates to methods for the creation of a cell monolayer comprising: a) providing a cell population; b) culturing said cell population in a liquid medium; and c) air-drying the cell population with a sample evaporator to create a cell monolayer; and optionally d) staining the air-dried sample with histological or other stains. The invention is particularly advantageous for use in high-throughput, high-content analysis of cell samples and, in particular, examining the effect of therapeutic agents on said cell samples.

Characterizing cells and cell populations in a biological study is an important step in understanding disease presence and/or progression. Cellular image analysis is used to investigate cells at the single-cell level which may provide crucial information on the cell microenvironment and cell population phenotypes, which in turn is highly useful for applications in medical diagnostics, cell-based assays, compound discovery and characterization such as stratifying patients during clinical trials. The field of high-content imaging provides compelling evidence that the analysis of cell-to-cell variability, cell microenvironment and cell-population phenotype is necessary for the determination of rare events and complete understanding of the population level phenotypes (reviewed in Snijder et al., Nature Reviews Molecular Cell Biology, 2011, 12, 119-125). Variations in cellular phenotype on a global level are largely determined by the inherent properties of developing cell populations that create specialized niche microenvironments, including cell densities, cell-cell contacts, relative location and cell-space. Moreover, at the single-cell level, the heterogeneity of cellular-responses to the same perturbation, such as a therapeutic agent, can be characterized (Slack et al., PNAS 2008, 105, 19306-11; Snijder et al., Nature, 2009, 461, 520-523). The complexity of cellular heterogeneity, for example, in reference to the investigation of degree to which cancer cells react to anti-cancer drugs, reveals functional significance to the broad effect patients may have on a cellular level during therapy.

However, studies which have laid the groundwork for population-characteristic analysis driven by subcellular and single-cell resolution have relied on genetically identical cell lines, which are not physiologically relevant to human health and disease and lack the intra-population communication that occurs normally within life to further cellular and pan-cellular processes. In this regard, methods of the prior art comprise separation/isolation of cell populations and/or other means, which destroy natural cell-cell interactions and/or membrane integrity, by, inter alia, application of gravitational forces, in particular centrifugation or spinning, or cell lysis (see, e.g., Douglas et al. (2001) Current Protocols in Immunology (May 1 2001); Katrien Princen et al. (2002) Cytometry Part A, 51A, no. 1, pp. 35-45; Peter Ambros et al. (2004) Current Protocols in Cytometry (Nov. 1 2004)). These methods do not reflect physiologically relevant states and/or are unable to analyze physiologically relevant cell samples. The same holds true for Cytospin® analyses, which uses gravitational forces to obtain cell samples, wherein almost no natural occurring cell-cell interactions are maintained (see e.g., Ikeda et al., Diagnostic Cytopathology, 2011, 39, 395-401).

Moreover, the characterization of cells and cell populations is limited by the fact that imageable cell monolayers typically comprise adherent cells (such as macrophages, HeLa, etc.), which require a surface, such as a tissue culture plastic, optionally coated with extracellular matrix components to increase adhesion properties and provide other elements needed for growth and differentiation. Most mammalian cells derived from solid tissues are adherent in nature. However, there are many applications where non-adherent mammalian cell culture is desirable, such as with embryonic stem cells, neural stem cells, and hematopoietic cells, e.g., cells derived from blood-based diseases such as lymphomas and leukemias. Furthermore, non-adherent cells (otherwise known as suspension cells) derived from cancer cells have been shown to better mimic primary tumor-like behavior, therefore their use is particularly advantageous for image-based single cell screening techniques in primary patient samples for high-throughput determination of chemotherapy-induced molecular (biomarker) changes and cancerous blast viability assessments.

Image-based single-cell drug testing and analysis may be of particular use for personalized diagnosis or personalized medicine, an emerging field which has the potential to revolutionize healthcare. To date, the state-of-the-art technology in personalized medicine is genomic molecular profiling which stratifies patients into subgroups based on genetic features with the aim to guide patient treatment selection. Although this method has been successfully applied to several diseases, its clinical benefit is yet to be realized. This is due to the large abundance of non-targetable genomic mutations, differential epigenetic regulation, differential drug penetrance and transport, as well as differences in the interactions between various cell types and tissues which makes every patient unique and complicates personalized treatments. Image-based drug testing offers a solution to the problems encountered in genomic profiling, by using the patients'own biopsied cells to directly test a large panel of drugs, wherein diseased and healthy cells can be simultaneously measured, allowing both drug efficacy and potential toxicity to be predicted for individual patients. This direct functional readout of cell cytotoxicity and changing cell phenotypes encapsulates not only the genomic profile of a person but also the epigenetic regulations, metabolic state and interactions between different cell types. The pharmacoscopy method, previously disclosed by part of the present inventors (Snijder et al., Lancet Haematol. 2017, 4, E595-E606) comprises single-cell drug testing combined with immunofluorescence and high-content imaging in primary patient samples. However, its reliance on fluorescence microscopy limits its use due to the need for sophisticated, fragile, expensive and slow equipment and extensive sample manipulation.

Many imaging methods rely on detecting and identifying cells using fluorescence-based immunohistochemistry methods, which are not well suited to the detection of malignant cells derived from patient samples. Fluorescent immunohistochemistry stains depend on the expression of selected proteins and therefore have limited applications due to, inter alia, the need to customize the stain for each disease type and at times for individual patients. Moreover, immunofluorescent stains commonly have problems related to low sensitivity, extensive sample preparation protocols, high background noise, autofluorescence and photobleaching. Furthermore, expensive equipment is typically required, such as automated liquid handling equipment and laser or LED based microscopes. A further issue with immunohistochemistry methods is the use of tissue samples that have been frozen in a cryopreservation medium (such as an optimal cutting temperature compound) or fixed in neutral buffered formalin and embedded in paraffin. Such procedures may damage various cellular components. Standard clinical procedures, utilize histological and hematological dyes to identify malignant cells based on their morphology. This serves as a more comprehensive marker for malignancies than individual fluorescent markers.

There is therefore a need in the art for high-content imaging methods which rely on stains that are readily available, easily manipulated and are more robust for clinical applications.

The following prior art documents are also identified herein:

Document WO2016046346A1 describes peripheral blood mononuclear cell (PBMC) monolayers or bone-marrow cell monolayers and methods for their culture and corresponding uses of said monolayers. The invention is also described to relate, in some aspects, to screening methods comprising the PBMC monolayer or bone-marrow cell monolayer of the invention for determination of response or lack of response of a disease to a therapeutic agent and/or drug screening methods. Furthermore, the invention is said to relate to methods for diagnosing a disease or predisposition to a disease in a PBMC donor or bone-marrow cell donor comprising the PBMCs/bone-marrow cells cultured according to the methods of the invention and/or to methods for determining whether the disease is likely to respond or is responsive to treatment with a therapeutic agent.

Document WO2018154072A1 describes methods for determining the propensity of cells to interact in a population of cells comprising at least two distinguishable subpopulations of cells. In addition, the document describes methods for diagnosing a disease or predisposition to a disease of a cell donor, wherein the method comprises the determination of the propensity of cells to interact in a population of cells obtained from the donor, wherein the population of cells comprises at least two distinguishable subpopulations of cells. Moreover, methods for determining whether a subject suffering from or predisposed to a disease will respond or is responsive to treatment with a therapeutic agent is described.

Document WO2017191203A1 describes a multiwell imaging plate and a method for incubating non-Adherent cells.

Rothfels et al. (1958) Stain Technology, 33:2, 73-77 describes a method to analyze chromosomes in mammalian cells grown in vitro. The method involves air drying of fixed cells on glass slides. Non-adherent cells are fixed in solution, pipetted on the slides and air dried before imaging.

Fulciniti et al. (2008) Acta Cytologica (2008) 52 (2): 178-186 shows a technique for performing immunocytochemical staining on cytologic samples that are air-dried and fixed.

Ifuji et al. (2018) MethodsX, pages 431-437 describes a simple method for immunofluorescence staining of microtubules on glass slides.

Eggerschwiler et al. (2018) Stem Cell Research & Therapy 10:69 uses either 24-well or 48-well plates with adherent cells and applies histological stainings.

There is a recognized need in the field for advanced high-content imaging methods that utilize commonly available hematological or histological stains. These methods should be easily manipulated and robust enough for clinical applications. Additionally, there is a necessity for imaging techniques that accurately reflect the in vivo state of cell samples. This involves creating cell monolayers using non-disruptive and gentle methods.

Furthermore, the effective use of these stains requires the removal of water prior to staining, typically achieved through thorough air drying. Consequently, there is a need for an efficient method of air drying cellular samples in multi-well plates, facilitating the use of these stains in high-content screening.

The present invention addresses this need and solves the problem of providing a novel and improved method for the creation of a cell monolayer which may be used in methods of diagnosis of a disease or predisposition to disease, in drug screenings, and in the assessment of treatment results or predispositions to treatment, particularly in high-throughput high-content imaging that is amenable to clinical settings.

Accordingly, the invention, inter alia, relates to the following embodiments:
1. A method for the creation of a cell monolayer comprising
   a) providing a cell population;
   b) culturing said cell population in a liquid medium; and
   c) air-drying the cell population with a sample evaporator to create a cell monolayer.
2. The method of item 1, wherein naturally occurring cell-cell interactions, cell/membrane integrity are maintained during the formation of the monolayer.
3. The method of item 1 or 2, wherein the cell population is incubated at a density of about 100 cells per mm² to about 30000 cells per mm².
4. The method of any one of items 1 to 3, wherein the cell population is obtained from a primary cell culture, a cell line or a cell strain, in particular from a primary patient sample or a combination thereof.
5. The method of any one of items 1 to 3, wherein the cell population is obtained from a cell donor.
6. The method of any one of items 1 to 5, wherein air-drying immobilizes the cells onto a surface.
7. The method of any one of items 1 to 6, wherein the cell population comprises non-adherent cells or a mixture of non-adherent and adherent cells.
8. The method of any one of items 1 to 7, wherein the sample evaporator supplies pressurized air at an air speed of 0.5 to 2.5 liters per minute per mm² (L/min/mm²), preferably 1.0 to 2.0 L/min/mm², more preferably 1.5 L/min/mm².
9. The method of any one of items 1 to 8, wherein air-drying is continued until the cell population is dry and the cell monolayer is created, preferably wherein the cell population is air-dried between 10 min and 12 hrs, more preferably between 10 min and 6 hrs.
10. The method of any one of items 1 to 9, wherein the temperature of the supplied air is between 10 to 50 C, preferably between 20 to 40 C, more preferably between 23 to 37 C.
11. The method of any one of items 1 to 10, wherein prior to air-drying the cell population, part of the liquid media is removed, preferably wherein the liquid media is removed until at least 1 mm of liquid media remains above the surface of the cell population.

### 12. the Method of Any One of Items 1 to 11, Wherein the Cells Are Cultured in a Multi-well Plate.

13. The method of any one of items 1 to 12, wherein the method further comprises a step of chemically fixing the cell monolayer.
14. The method of any one of items 1 to 13, wherein the method further comprises, subsequent to step (c), a step (d) comprising staining the monolayer.
15. The method of item 14, wherein said staining involves staining with a histological stain.
16. The method of item 15, wherein the histological stain is a hematoxylin and eosin stain (H & E stain), hematoxylin stain, eosin stain, Giemsa stain, Pappenheim stain, May-Gruenwald stain, methylene blue stain, azure B stain, Masson triple or trichrome stain, aldehyde fuchsin stain, Verhoeff-van Gieson elastic tissue stain, silver stain, Periodic acid-Schiff stain, Gram stain, Papanicolau's (Pap) stain, Immunoperoxidase stain or a combination thereof.
17. The method of item 14, wherein said staining involves staining with a fluorescent stain.
18. The method of item 15 or 16, wherein subsequent to staining the monolayer with a histological stain, the method further comprises a step of staining the monolayer with a fluorescent stain.
19. A method for determining whether a cell donor suffers from a disease, the method comprising
   a) providing the cell monolayer created by the methods of any one of items 5 to 16;
   b) analyzing the cell monolayer; and
   c) determining whether the cell donor suffers from a disease.
20. The method of item 19, wherein the disease is an infectious disease, solid cancer or liquid cancer.
21. The method of item 19 or 20, wherein said solid or liquid cancer is a carcinoma, sarcoma, myeloma, leukemia, lymphoma or a mixed-type cancer.
22. The method of any one of items 19 to 21, wherein said solid cancer or liquid cancer is glioblastoma (GBM) or acute myeloid leukemia (AML).
23. The method of item 19 or 20, wherein the infectious disease is caused by a pathogen, such as a bacterium, fungus, parasite or virus.
24. A method for determining whether a cell donor suffering from a disease will respond or is responsive to treatment with a therapeutic agent comprising
   a) providing a cell population;
   b) culturing said cell population in a liquid medium comprising the therapeutic agent;
   c) air-drying the cell population with a sample evaporator to create a cell monolayer;
   d) imaging the cell monolayer created in step (c); and
   e) assessing the response of the cells in the cell monolayer to the therapeutic agent to determine whether the cell donor suffering from a disease will respond or is responsive to treatment with the therapeutic agent.
25. The method of item 24, wherein said assessment in step (e) comprises applying a computer-implemented image analysis, such as an artificial neural network, for the analysis of the cell monolayer, the analysis comprising
   i) detecting cell locations by object detection;
   ii) determining one or more cellular characteristic(s) in the cell monolayer; and
   iii) determining the response of the cells in the cell monolayer to the therapeutic agent, based on the one or more cellular characteristic(s) determined in step (ii).
26. The method of item 24 or 25, wherein steps (a) to (e) are repeated for different concentrations of the therapeutic agent to determine an optimal concentration.
27. The method of any one of items 24 to 26, wherein the one or more cellular characteristic(s) comprise(s) cell viability, cell morphology, cell-cell interactions, membrane integrity, the identification or measurement of a biomarker and/or the presence of a pathogen or a combination thereof.
28. The method of any one of items 24 to 27, wherein the therapeutic agent is assigned a drug response score based on the response of the cells and/or cell population(s) as determined by the one or more cellular characteristic(s).
29. The method of item 28, wherein the disease is a solid or liquid cancer and wherein the drug response score is determined by calculating the number of cancer cells determined in step (ii) divided by the average number of cancer cells in a control sample, divided by the number of non-cancer cells determined in step (ii), divided by the average number of non-cancer cells in a control sample.
30. The method of any one of items 24 to 29, wherein the method further comprises staining the monolayer subsequent to air-drying the monolayer with the sample evaporator in step (c).
31. the Method of Item 30, Wherein Said Staining Involves Staining With a Histological Stain.
32. The method of item 30 or 31, wherein the histological stain is a hematoxylin and eosin stain (H & E stain), hematoxylin stain, eosin stain, Giemsa stain, Pappenheim stain, May-Gruenwald stain, methylene blue stain, azure B stain, Masson triple or trichrome stain, aldehyde fuchsin stain, Verhoeff-van Gieson elastic tissue stain, silver stain, Periodic acid-Schiff stain, Gram stain, Papanicolau's (Pap) stain, or Immunoperoxidase stain or a combination thereof.
33. the Method of Item 30, Wherein Said Staining Involves Staining With a Fluorescent Stain.
34. The method of item 31 or 32, wherein subsequent to staining the monolayer with a histological stain, the method further comprises a step of staining the monolayer with a fluorescent stain.
35. A method for selecting a therapeutic agent for use in the treatment of a disease of a subject having said disease, wherein said therapeutic agent is selected from at least two or more test agents, wherein each of the at least two or more test agents is tested in an assay comprising
   a) providing a cell population;
   b) culturing said cell population in a liquid medium comprising the test agent;
   c) air-drying the cell population with a sample evaporator to create a cell monolayer;
   d) staining the cell monolayer, preferably with a histological stain;
   e) imaging the cell monolayer created in step (d); and
   f) assessing the response of the cell monolayer by applying a computer-implemented image analysis for the image obtained in step (d), said analysis comprising
      i) detecting cell locations by object detection;
      ii) determining one or more cellular characteristic(s) in the cell monolayer; and
      iii) determining the response(s) of the cell population(s) in the cell monolayer to the test agent based on the one or more cellular characteristic(s) determined in step (ii),
         wherein the assay is repeated for each of the at least two or more test agents and wherein each of the test agents is assigned a drug response score based on the response(s) of the cell population(s) in the cell monolayer as determined by the one or more cellular characteristic(s) identified in step (ii).
36. The method of item 35, wherein the cellular characteristic comprises cell viability, cell-cell interactions, cell morphology, membrane integrity, the identification or measurement of a biomarker and/or the presence of a pathogen or combination a thereof.
37. the Method of Any One of Items 1 to 36, Wherein the Method Is Fully Automated.
38. Use of a monolayer created by the methods of any one of items 5 to 18 for determining whether a cell donor suffers from a disease.
39. Use of a monolayer created by the methods of any one of items 5 to 18 for determining whether a subject suffering from a disease will respond or is responsive to treatment with a therapeutic agent.
40. The use of item 38 or 39, wherein the disease is an infectious disease, a solid cancer or liquid cancer.
41. The use of any one of items 38 to 40, wherein said solid or liquid cancer is carcinoma, sarcoma, myeloma, leukemia, lymphoma or a mixed-type cancer.
42. The use of any one of items 38 to 41, wherein said solid or liquid cancer is glioblastoma (GBM) or acute myeloid leukemia (AML).
43. A culture device comprising a monolayer created by the methods of any one of items 1 to 37, wherein the culture device comprises a multi-well plate.
44. The culture device of item 43, further comprising a sample evaporator.
45. The culture device of item 44, wherein said sample evaporator comprises a plurality of air outlets located above and/or partially within the multi-well plate.
46. The culture device of any one of items 43 to 45, wherein the multi-well plate comprises flat-bottom wells.

Some of the embodiments of the invention are reflected in the figures as part of this disclosure.

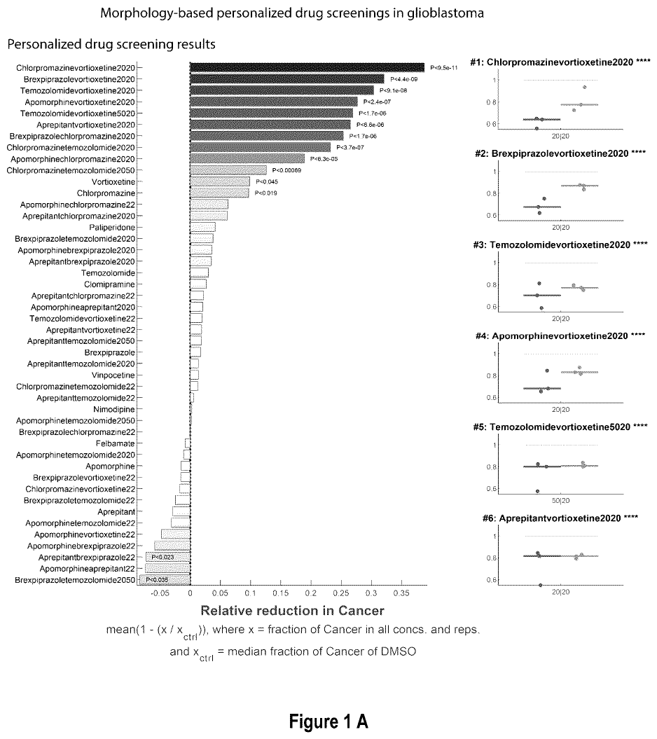

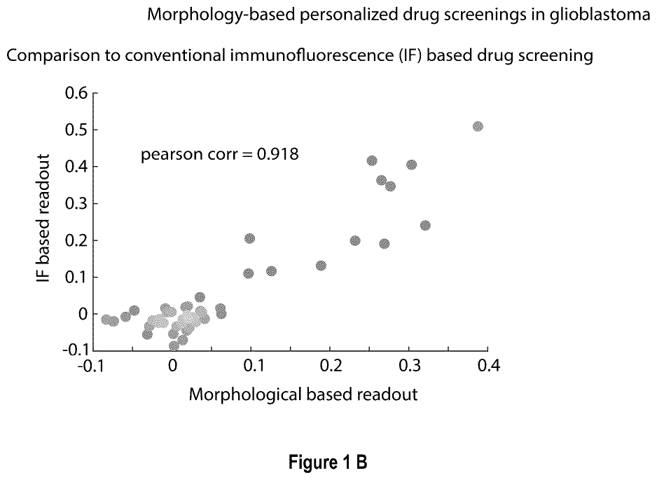

FIG. 1: A) Depicts the outcome of the personalized drug-screening using the methods of the invention with a monolayer comprising PBMCs co-cultured with cells from a glioblastoma tumor sample obtained from a cell donor suffering from glioblastoma. B) Compares the readout from the current method with those obtained from conventional immunofluorescence-based drug screening on the same sample.

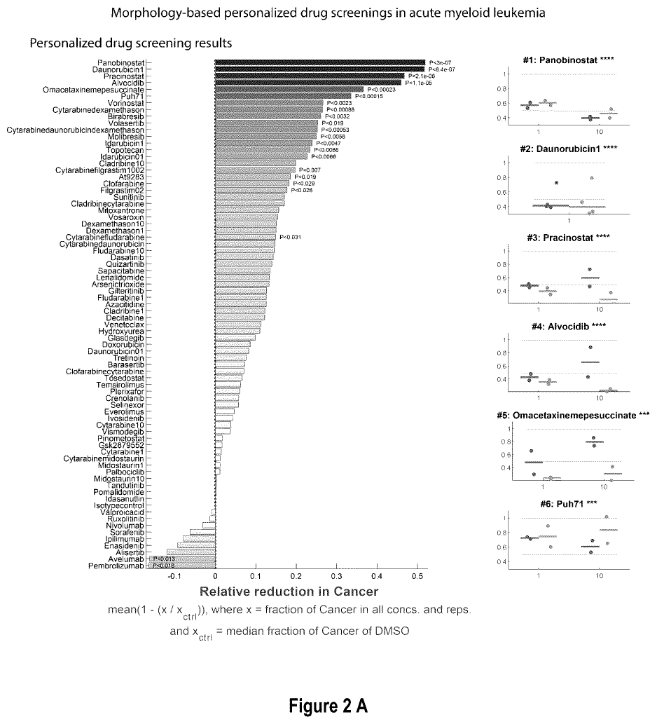

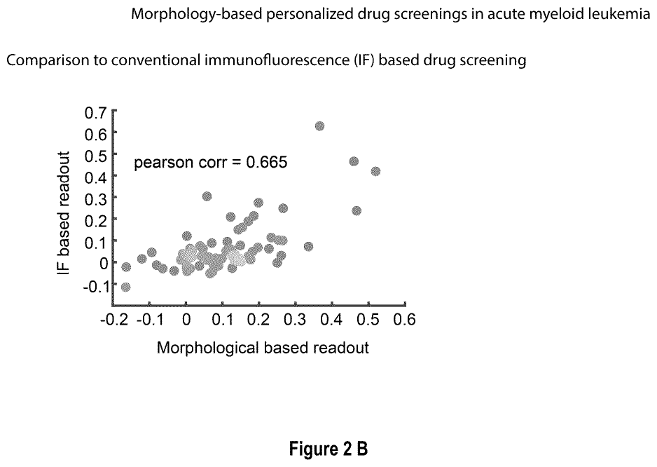

FIG. 2: A) Depicts the outcome of the personalized drug-screening using the methods of the invention with a cell monolayer obtained from a cell donor suffering from acute myeloid leukemia. B) Compares the readout from the current method with those obtained from conventional immunofluorescence-based drug screening on the same sample.

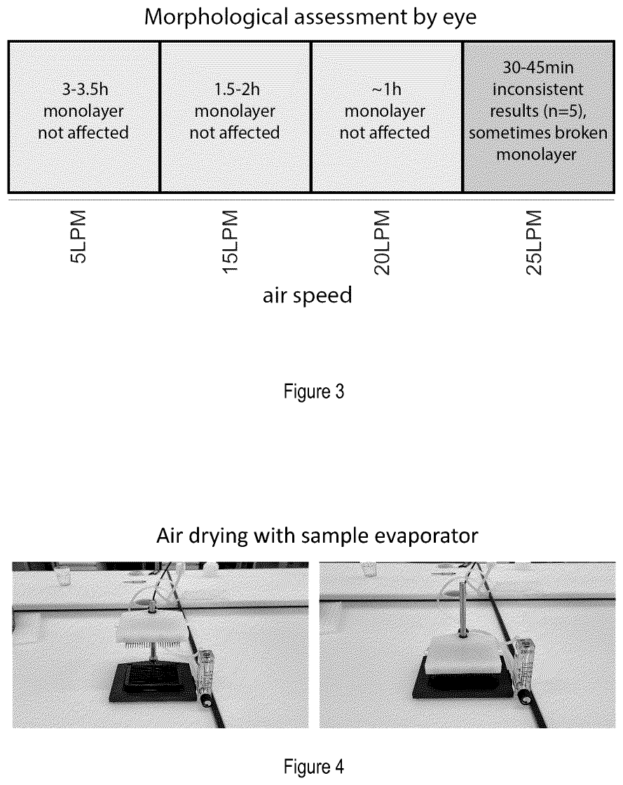

FIG. 3: Depicts the morphological assessment by eye on monolayers comprising lymphocytes at different air speeds of the sample evaporator.

FIG. 4: Depicts a cell culture device comprising a 384-well plate with an automated sample evaporator placed directly above the well plates, wherein the height of the sample evaporator may be adjusted.

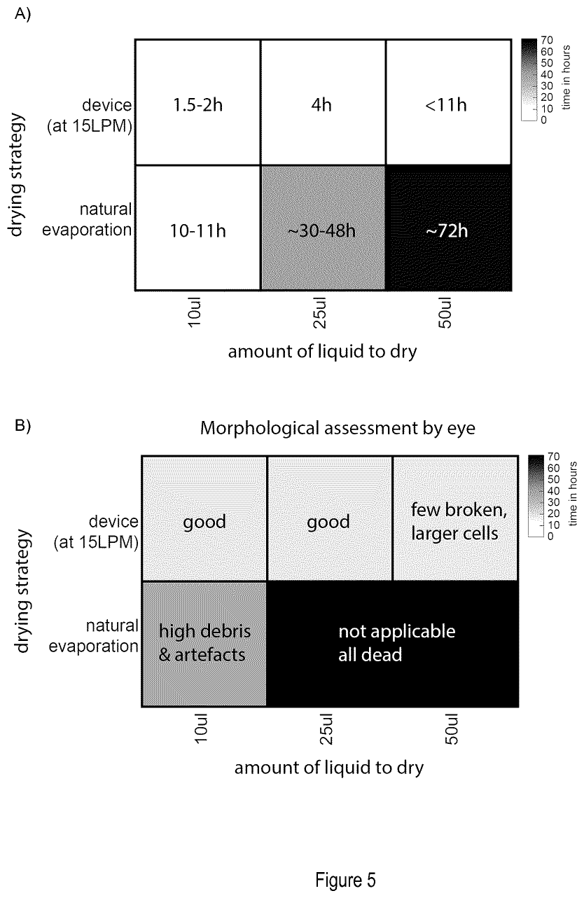

FIG. 5: Depicts a comparison of the air-drying method of the invention and natural evaporation. A) Compares the amount of time taken to dry the cell monolayer using the air-drying method and natural evaporation. B) Compares the morphological assessment of the samples after air-drying and natural evaporation.

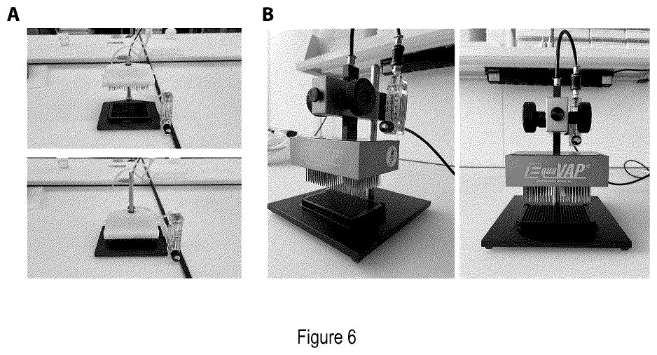

FIG. 6: Tested sample evaporator used for monolayer creation of non-adherent cells. A) Cole-Parmer Evaporator/Concentrator for 384-well Plates, #EW-28690-55. B) Analytical EquaVAP 384-Well Evaporator, #23384.

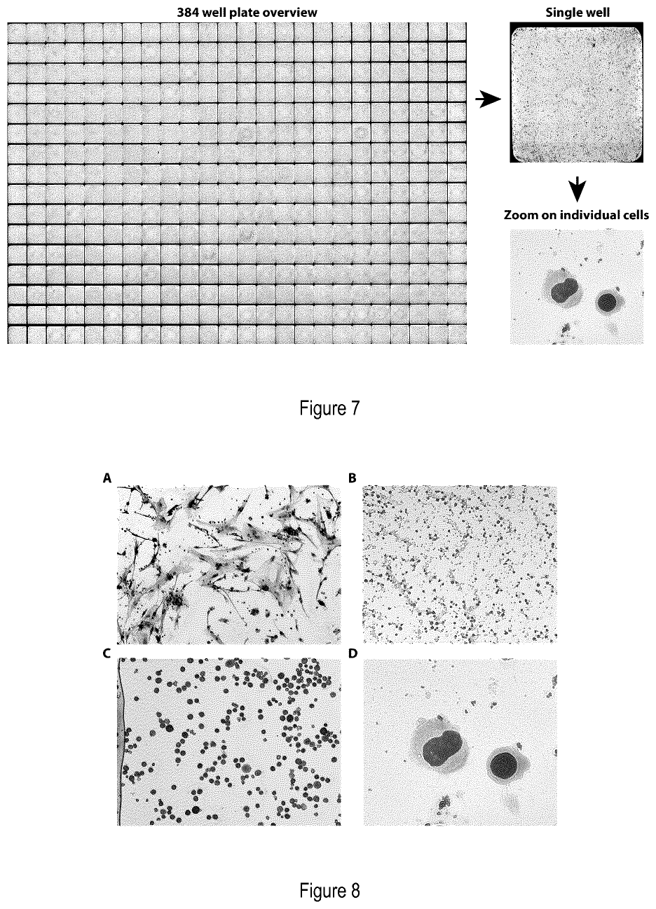

FIG. 7: Cells isolated from a primary AML patient sample and seeded in a 384-well containing different medications. Samples were air-dried for monolayer creation and stained with a Pappenheim staining. Images were generated with a EVOS M7000 automated microscope in color imaging mode. Zoom in was imaged at a 40× resolution.

FIG. 8: Usage of different staining's and samples after monolayer creation by air drying in a 384 well plate. A) Primary GBM cancer cells mixed with healthy PBMCs stained with a Pappenheim staining, 20×. B) Healthy PBMCs stained with a Wright-Giemsa staining, 20×. C) Ramon B lymphocyte cell line staining with Giemsa, 20×. D) Primary AML blast stained with Pappenheim, 40× zoom in.

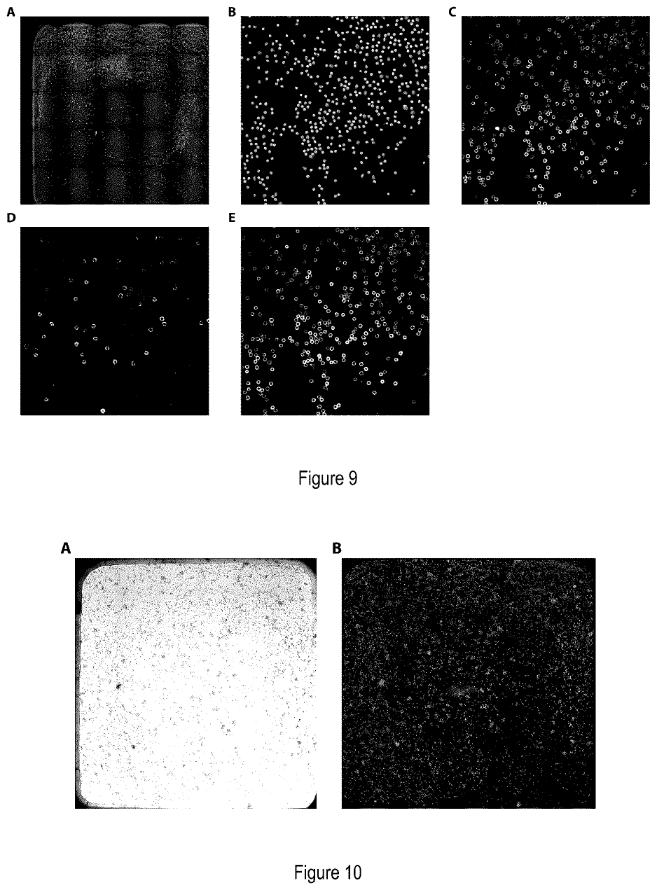

FIG. 9: Fluorescent staining of primary PBMC after monolayer creation with air-drying, 20× resolution A) Overview of single well. B) DAPI staining of a single site. C) CD4 staining of a single site. D) CD8 staining of a single site. E) CD3 staining of a single site.

FIG. 10: Monolayer creation by air-drying is compatible with simultaneous hematological and fluorescent staining. A) Giemsa-Wright stain of primary AML cells B) DAPI staining of the same matching cells.

In a first embodiment, the invention relates to a method for the creation of a cell monolayer comprising: a) providing a cell population; b) culturing said cell population in a liquid medium; and c) air-drying the cell population with a sample evaporator to create a cell monolayer.

The methods and means provided herein may advantageously make use of widely applicable and inexpensive stains, thus allowing the use of the cell monolayers in methods of diagnosis of a disease or predisposition to disease, in drug screenings, and in the assessment of treatment results or predispositions to treatment.

The term “air-drying”, as used herein, refers to drying in an airstream by blowing air onto the cell population using a “sample evaporator” such that the cells will appear to be dried and a cell monolayer is created. The time required to dry the cells depends on the strength of the airstream and the distance between the sample evaporator and the surface of the media comprising the cell population. The sample evaporator of the present invention may be any apparatus or device which comprises an element that is designed to provide an airstream from pressurized air via an air flow means. As is evidenced by the appended Examples and Figures, air-drying of the cell population using a sample evaporator reduces cell loss and significantly reduces the time taken to create a cell monolayer when compared with natural evaporation methods. The inventors found that allowing the cell population to dry by natural evaporation took 10-72 hrs of drying for 10-50 μL of liquid media and in all cases led to cell loss, due to apoptosis for example. When natural evaporation was continued for more than around 30 hrs, most cells were lost due to apoptosis. Conversely, using a sample evaporator for air-drying, the inventors found that the drying time is significantly reduced to 1.5-11 hrs for 10-50 μL of liquid media and cells remained morphologically intact by eye for samples dried for less than around 4 hrs. At around 11 hrs of air-drying with the sample evaporator, minor morphological changes in the cell monolayer could be observed. Therefore, the air-drying protocol of the present invention provides a method and means for retaining the in vivo state of cell populations and is a fast and simplistic protocol for the creation of a monolayer, which is applicable to clinical settings and high-throughput applications such as drug screening.

As is well understood in the art, the creation of cell monolayers, in particular monolayers of non-adherent cells or mixed non-adherent and adherent cells typically require careful preparation and monitoring, especially when manipulating media to avoid disruption such that the sample maintains an in vivo state. It is common in methods of the prior art for disturbances, e.g., vortexes and turbulence in the medium, to occur when removing/replacing media using, for example, manual or automated aspirators which may result in, inter alia, cell loss and/or changes in cell-cell interactions and/or morphology. Moreover, certain non-adherent cell lines, such as lymphoma and leukemia cell lines, may attach poorly to surfaces using currently available means and methods. The present inventors surprisingly found that instead of chemically fixing and imaging cells in liquid, a monolayer can be created by air-drying the cell population using a sample evaporator such that the in vivo state of the cells is preserved. The inventors found that air-drying the cell population using a sample evaporator forces the cells to the bottom of a culture surface, thus immobilizing and fixing the cells to the surface and creating a cellular monolayer. The cell monolayer produced by this method reflects the in vivo characteristics of the cell population and may be used for imaging and drug screening purposes as well as in many other applications as can be determined by a person skilled in the art.

The “cell population” used in the methods of the present invention is not particularly limited and refers to any cell sample obtained from a biological organism, preferably a living organism. The cell population may also be derived from non-living, i.e. deceased organisms, in particular recently deceased organisms. Furthermore, the cell population may also comprise single cell organisms. In preferred embodiments of the invention, the cell population may be derived from an animal, preferably from a mammal, more preferably from a human. In specific embodiments of the methods of the present invention, the cell population is a sample of non-adherent cells or a mixture of non-adherent and adherent cells. In a further preferred embodiment of the invention, the cell population comprises a sample of primary hematopoietic cells, in particular peripheral blood mononuclear cells (PBMCs) or bone-marrow cells. PBMCs and bone-marrow cell samples, comprise, inter alia, cells within the following groups of cells, and cells within the lineage of the cells, including terminal cell states: Hematopoietic stem cells (including, but not limited to, common lymphoid progenitor, common myeloid progenitor, and their maturation lineage and terminal states including pro-B-cell, B-cell, double negative t-cells, positive T-cell, plasma-B-cell, NK-cells, monocytes (macrophage, dendritic cells)). These can be found, but not limited to, within peripheral blood, bone marrow (flat bone localized), cord blood, spleen, thymus, lymph tissue, and any fluid buildup result of a disease such as pleural fluid. Cells may be in any healthy or diseased state.

Within the present invention, the term “cell population” is used interchangeably with the term “cell subpopulation”. the skilled person is well-aware that a cell sample or cell population, in particular the monolayer of the invention, comprises cells of different subpopulations, wherein each cell of each subpopulation may be in a state of, inter alia, living, dead and/or dying. That is, in the monolayers of the present invention, the number/ratio of cells in each state, i.e. living, dead or dying, and each subpopulation preferably corresponds to the number/ratio that is found in vivo. That is, the physiologically-relevant state in which cells comprised in the monolayer of the present invention are found is preferably devoid of cells, which are found in a different state, i.e. living, dead or dying, as they are found in vivo or belonging to a different subpopulation. This requires that the preparation of the monolayer provided herein does not alter the number/ratio of cells in each state and/or in each subpopulation found in vivo.

Accordingly, the methods of the present invention are advantageously able to maintain numbers/ratios of cells of each subpopulation, since pipetting is reduced during the formation of the cell monolayer. In this regard, samples used herein comprise adherent and non-adherent cells. The method of the present invention overcomes issues encountered by methods in the prior art by reducing the removal of non-adherent cells by pipetting or other liquid medium manipulation techniques prior to the formation of the monolayer, thus maintaining the overall representation of subpopulations comprised in the sample to be used for monolayer formation.

In contrast, Fulciniti et al. and Ifuji et al. disclose distinct methods. In particular, Fulciniti uses air-drying and formalin fixation, specifically ethanol wet-fixed and air-dried smears, for immunocytochemical staining on slides, while Ifuji describes a method to air-dry cultured cells using a hair dryer, fixation and immunofluorescence staining.

Peripheral blood mononuclear cells (PBMCs) are blood cells having a round nucleus (as opposed to a lobed nucleus). PBMCs comprise lymphocytes (B-cells, T-cells (CD4 or CD8 positive), and NK cells), monocytes (dendritic cell and macrophage precursor), macrophages, and dendritic cells. These blood cells are a critical component in the immune system to fight infection and adapt to intruders. PBMCs cells for use in the methods described herein can be isolated from whole blood using any suitable method known in the art or described herein. For example, the protocol described by Panda et al. may be used (Panda, S. and Ravindran, B. (2013). Isolation of Human PBMCs. Bio-protocol 3(3): e 323). Preferably, density gradient centrifugation is used for isolation. Such density gradient centrifugation separates whole blood into components separated by layers, e.g., a top layer of plasma, followed by a layer of PBMCs and a bottom fraction of polymorphonuclear cells (such as neutrophils and eosinophils) and erythrocytes. The polymorphonuclear cells can be further isolated by lysing the red blood cells, i.e. non-nucleated cells. Common density gradients useful for such centrifugation include, but are not limited to, Ficoll (a hydrophilic polysaccharide, e.g., Ficoll®-Paque (GE Healthcare, Upsalla, Sweden) and SepMate™ (StemCell Technologies, Inc., Köln, Germany).

Bone-marrow cells for use in the methods described herein can be isolated from bone marrow using any suitable method known in the art. In particular, density gradient centrifugation and magnetic beads can be used to separate bone-marrow cells from other components of such samples. For example, MACS cell separation reagents may be used (Miltenyi Biotec, Bergisch Gladbach, Germany).

As is known in the art, such isolated cultures may contain a small percentage of one or more populations of another cell type, e.g., non-nucleated cells such as red blood cells. The cells, such as PBMCs, may be further isolated and/or purified from such other cell populations as is known in the art and/or as described herein; for example, methods of lysing red blood cells are commonly used to remove such cells from isolated PBMCs. However, the methods of the invention are not reliant on further purification methods, and the isolated cells, including PBMCs, isolated herein may be directly used. Accordingly, the methods disclosed herein may or may not comprise lysing of red blood cells from within the sample of isolated PBMCs. However, where present, it is believed that the presence of non-nucleated cells, e.g., red blood cells, being generally smaller than PBMCs, settle on the culture surface below and between the PBMCs, and potentially interfere with the formation of a monolayer suitable for imaging. Therefore, it is preferred that the concentration of non-nucleated cells, e.g., red blood cells, in the cell monolayer is less than 10%, less than 5%, less than 1% or less than 0.1%.

In a further preferred embodiment of the invention, the cell population comprises a sample of primary cells derived from solid tumors, including but not limited to glioblastoma and other primary brain tumors (such as glioma, ependymomas, medulloblastomas and oligodendrogliomas), breast cancer, skin cancer, colorectal cancer, ovarian cancer, testicular cancer, and other cancers. Cells obtained from such tumor samples can include both adherent and non-adherent cells.

The term “culturing”, “cell culture”, “incubation” and grammatical variations thereof are used interchangeably herein and refer to the process by which cells are grown under controlled conditions, typically outside of their natural environment. Cell culture methods are well-known in the art, for example, methods described by Panda et al. may be used (Panda, S. and Ravindran, B. (2013), In vitro Culture of Human PBMCs, Bio-protocol 3(3):e322). The culture conditions are defined by culture media, supplements, matrices, technically supported micro-environment and gas supply. Individual culture units may provide comparable conditions. The culture conditions may be chosen according to the type of cells. For example, cells may be incubated at 37 °C, 5% CO₂ and 20% oxygen. The cultured cells may be provided with gas through a gas permeable membrane that seals at least one side of the culture well.

Preferably, within the methods of the present invention, cells are incubated at 37 °C in a CO₂ incubator with 5% CO₂. It is preferred to use RPMI as culture medium. The duration of the incubation step of the methods of the invention is not particularly limited. However, as recognized in the art, fragile cell populations, such as PBMCs, are difficult to maintain over the long term; culturing times of over 36 hours are likely to result in cell death. Accordingly, where incubation times of over 36 hours are to be used, the health of the culture should be monitored to ensure that the monolayers remain viable. Typically, cultures are cultured for between 1 and 24 hours (e.g., overnight) which ensures that the monolayers remain viable. However, formation of an imageable cell monolayer, which may or may not be stained according to methods known in the art and/or described herein (comprising both adherent and non-adherent subpopulations of cells) is typically accomplished after short incubation, e.g., less than 1 hour. Where the cell monolayers are used in methods of the invention comprising the assessment of response to a therapeutic agent, longer incubation periods may be required to allow the agent to exert its activity and/or for an observable effect to be achieved (however, the cells must remain viable during this incubation, and, as explained herein, these longer incubation periods typically are at most 24 hours). Furthermore, the cell monolayer may comprise both, healthy and diseased cells which can subsequently be used for further analysis. Culturing does not imply any necessary minimal time; a cell population isolated from a sample, placed in a cell culture device and imaged are considered to be cultured on or in the device regardless of the length of time the cells have been on or in the device.

In a second embodiment, the invention relates to a method wherein naturally occurring cell-cell interactions and/or cell/membrane integrity are maintained during the formation of the monolayer.

The term “cell-cell interactions” as used herein refers to the direct interactions between cell surfaces that play a crucial role in the development and function of multicellular organisms. These interactions allow cells to communicate with each other in response to changes in their microenvironment. Such cell-cell interactions can be stable such as those made through cell junctions. These junctions are involved in the communication and organization of cells within a particular tissue. Others are transient or temporary such as those between cells of the immune system or the interactions involved in tissue inflammation. These types of intercellular interactions are distinguished from other types such as those between cells and the extracellular matrix.

Maintaining cell-cell interactions means that a cell interacting with another cell in a natural environment will also interact with said other cell or a cell of the same cell type in the monolayers provided herein. That is, the overall cell-cell interactions are maintained, while cells do not necessarily maintain interaction with the same interacting cell.

The cell-cell interactions are maintained during the formation of the monolayer of the invention. Generally, cell-cell interactions occur in the cell population, comprising, e.g., PBMCs or bone-marrow cells, of the invention. These natural-occurring cell-cell interactions are maintained during the subsequent production steps of the in vitro produced monolayer of the invention. In particular, the cell-cell interactions are maintained during addition of detectable labels and/or dyes, in particular a viability dye.

The skilled person is aware of methods how to determine/assess/track/verify cell-cell interactions, in particular how to distinguish between natural-occurring cell-cell interactions and those introduced during the preparation of a cell sample. As such, the skilled person understands that cells of the same type and/or cells of different types interact in a living organism. The majority of cells comprised in the monolayers of the present invention maintain their natural-occurring cell-cell interactions. That is, the majority of cells comprised in the monolayers of the present invention interact with the same cell or a cell of the same cell type as in vivo.

This distinguishes the monolayers of the present invention from known cell samples, which show random interactions of cells and/or cell-cell interactions, which are generally not found in vivo, e.g. cell clumping. Accordingly, the monolayers of the present invention reflect a physiologically-relevant state.

The term “cell/membrane” integrity refers to the cell and/or membrane integrity. During the formation of the monolayers provided herein, membrane integrity is maintained. This means that biological membranes as they occur in a natural environment are maintained during the process of forming the monolayer of the invention. Many of the above-described cell-cell interactions depend on intact membranes. Therefore, in the process of forming the monolayer of the invention, membrane integrity is preferably maintained. This may be achieved by avoiding the use of buffers that have an impact on membrane integrity, e.g. buffers interacting with biological membranes such as buffers comprising detergents. For example, buffers comprising Triton or SDS are to be avoided. The maintenance of membrane integrity during formation of the monolayer of the invention leads to formation of a monolayer comprising cells being in a physiologically-relevant state. Air drying also represents a crucial step to maintain membrane integrity during subsequent staining and fixation steps.

Inadequate drying prior to fixation can lead to strong cellular artifacts, like nuclear leakage or cellular changes. The presence of water traces in the methanol fixative and methanol containing histological/haematological dyes can also induce unintended changes. All this can potentially affect morphological assessments, cell classification, and consequently, the diagnosis. As outlined in “Blood Cells—A Practical Guide, Fifth Edition” by Barbara J. Bain, it's crucial to ensure cells are thoroughly dried before staining to attain optimal results.

In this regard, the person skilled in the art is well-aware of methods of assessing whether a cell sample, as for example the monolayer of the present invention, represents a “physiologically-relevant state” of the cells comprised in said cell sample. In this regard, the term “physiologically-relevant state” or similar terms as used herein refers to a state resembling/reflecting an in vivo situation as it is found in a living organism, wherein said organism may be healthy or diseased. Accordingly, the majority of cells comprised in the monolayer of the present invention preferably are in a state, which reflects the in vivo situation with regard to the stadium during a life cycle of cells and/or cell-cell interactions, as described above. As such, the skilled person is well-aware that a cell sample, in particular the monolayer of the invention, comprises cells being, inter alia, in a state of living, dead and/or dying. That is, in the monolayers of the present invention, the number/ratio of cells in each state, i.e. living, dead or dying, preferably corresponds to the number/ratio that is found in vivo. That is, the physiologically-relevant state in which cells comprised in the monolayer of the present invention are found is preferably devoid of cells, which are found in a different state, i.e. living, dead or dying, as they are found in vivo. This requires that the preparation of the monolayer provided herein does not alter the number/ratio of cells in each state. Therefore, it is preferred that gravitational force applied during preparation of the monolayer, i.e. by the methods of the present invention, does not exceed 1 g. Preferably, this is achieved by avoiding the use of centrifugation. Accordingly, the methods of the present invention preferably do not comprise centrifugation subsequent to isolation of the cells. In addition, it is preferred that the monolayer of the invention is kept in solutions/buffers, which allow the maintenance of a physiologically-relevant state. As such, membrane integrity is preferably maintained in the monolayers of the invention, i.e. cells are preferably not lysed during preparation of the monolayer of the invention. In line with the above, the majority of cells comprised in the monolayers of the invention are preferably in a physiologically-relevant state, i.e. where cell-cell interactions are maintained and/or membrane integrity is maintained.

The “majority of cells” as used herein means that at least 50% of the cells comprised in the monolayers of the invention, preferably 60%, 65%, 70%, 75%, 80%, 85%, 90%, 95%, or 100% of the cells comprised in the monolayers of the invention are found in a physiologically-relevant state. The above percentages of cells in a physiologically-relevant state comprised in the monolayers of the invention are determined/measured/assessed using methods well-known in the art. In particular, whether a cell sample, in particular the monolayer of the present invention, comprises cells found in a physiologically-relevant state is determined by quantification of cells comprised in the monolayer. This may be done using methods well known in the art. In particular, quantification may be done through image analysis, preferably a computer-implemented image analysis, and a comparison may be made in cells of a reference individual or multiple reference individuals, e.g. one or more healthy donor(s) where the cell sample is derived from a diseased donor. Quantification of cells is a standard diagnostic tool. Thresholds of cell subpopulations, for example comprised in PBMCs and/or bone-marrow cells, are well typically documented for healthy donors and diseased donors. Accordingly, based on differences in samples to be assessed using the means and methods of the present invention, the physiological-relevance can be determined. Documentation of cell subpopulations comprised in hematopoietic cells can be found, for example, in Hallek et al., Blood, 2008, 111, 5446-5456. Accordingly, quantification and further means and methods, for example determination of cell-cell interactions using microscopy and/or a computer-implemented image analysis, allow the determination whether a cell sample represents a physiologically-relevant state.

The present invention relates to an in vitro produced cell monolayer, wherein natural-occurring cell-cell interactions and membrane integrity are maintained during formation of the monolayer. This may be achieved where cells are maintained/processed/analyzed at about 1g, i.e. 9.81 m/s² during the formation of the monolayer. In this respect, 1 g corresponds to standard gravity on the planet Earth, i.e. about 9.81 m/s². This includes that during the process of formation of the monolayer of the invention, centrifugation and/or spinning are avoided, i.e. the sample is not subject to centrifugation and/or spinning.

In some aspects, the monolayer may provide a unique model system that can be used, inter alia, in biological, biochemical and biophysical research. Moreover, the monolayer can be used in medical diagnostic and screening methods, e.g. in automated medical diagnostic and screening methods. Certain monolayers provided herein require minimal donor material; thus, standard amounts of donor material, e.g., as obtained for routine blood analysis protocols, may be used to test or analyze greater numbers of perturbations (e.g., individual test conditions) per donation than is possible using current methods known in the art. Additionally, certain monolayers provided herewith can allow for the rapid assessment of results using imaging-based analysis, e.g., via assessment of microscopic images of stained samples (e.g., fluorescent staining via tagged antibodies), leading to automated processing, substantially reducing manpower requirements and processing/analysis times. It is to be understood that within the methods of the present invention, wherein the naturally occurring cell-cell interactions, cell/membrane integrity are maintained during the formation of the monolayer, this may not be the case if the cells are cultured or incubated with a cytotoxic agents (also referred to as antineoplastic agents) or other therapeutic agents which effect the cellular characteristics. Said cytotoxic agents are typically toxic to cells, preventing their replication or growth and are often used to treat cancer, rheumatoid arthritis and multiple sclerosis.

In a further embodiment, the invention relates to a method wherein the cell population is incubated at a density of about 100 cells per mm² to about 30000 cells per mm².

Accordingly, in certain aspects, the present invention provides methods for culturing or incubating cell populations in the form of a monolayer comprising (a) isolating a cell population from a sample and (b) incubating said cell population or said cell monolayer at a specific density. In particular, the density is that which maintains the cell monolayer culture during the entire culture time of the monolayer, e.g., from introduction into the culture device until final processing, e.g. prior to imaging. For example, the maximum density is such that the total number of cells (a) introduced into the culture device or (b) expected to be present in the culture device subsequent to culturing and prior to processing for imaging does not exceed that number present at maximum density for the cell monolayer as described herein. The densities of the invention are typically lower than would normally be seeded into wells for the cultivation of cells as known in the art. The cell populations of the invention may be introduced and/or cultured to have in the culture device a density of about 100 cells per mm² growth area to about 30000 cells per mm² growth area. More preferably, the cell populations are incubated at a density of about 500 cells per mm² growth area to about 20000 cells per mm² growth area, about 1000 cells per mm² growth area to about 10000 cells per mm² growth area, about 1000 cells per mm² growth area to about 5000 cells per mm² growth area, or about 1000 cells per mm² growth area to about 3000 cells per mm² growth area. Most preferably the PBMCs are incubated at a density of about 2000 cells per mm² growth area.

The present invention provides, in certain aspects, methods for culturing or incubating primary hematopoietic cells, in particular bone-marrow cells or PBMCs, in the form of a monolayer.

Incubation or culturing is carried out in a liquid medium. A person skilled in the art is well aware of suitable methods to maintain viability of cells, such as PBMCs or bone-marrow cells. However, the liquid medium to be used in the methods of the invention is not particularly limited. In this regard, medium stands for liquids with nutrients and substances necessary for cultivation of cells. Liquid culture media for culturing eucaryotic cells are known to the person skilled in the art (e.g., DMEM, RPMI 1640, etc). Suitable media may be selected depending on the type of cells to be cultured. For example, PBMCs or bone-marrow cells may be cultivated in RPMI 1640 10% FCS. Any suitable media may be chosen, however, media components should be selected that are known to not artificially influence PBMC response and/or bone-marrow cell response. Supplements describe substances to be added to culture media in order to induce or modify cell function (e.g. cytokines, growth and differentiation factors, mitogens, serum). Supplements are known to the person of skill in the art. One example of a serum commonly used with eukaryotic cells is fetal calf serum. The culture media may further be supplemented with antibiotics, such as penicillin, streptomycin, ciprofloxacin etc. In one embodiment, test substances and/or stimulatory agents may be added to living cell material in each individual unit separately. Test substances may be pharmaceutical drugs or drug components. Stimulators may comprise any of the substances which support maintenance, growth or differentiation of cells. In a particular embodiment, stimulators are substances which act on immune cells, e.g. by activation of immune cells. Stimulators for activation of immune cells are known from the prior art. Such agents may be polypeptides, peptides or antibodies and other stimulators. For example, OKT-3, interferon-alpha, interferon-beta and interferon-gamma, oligoCPGs, mitogens (e.g. PWM, PHA, LPS), etc. Test substances and stimulators may be injected into the cell culture medium. Preferably, PBMCs are cultured in RPMI supplemented with FBS/FCS at 10% (preferably but not necessarily having low endotoxin raitings to minimize activation). PBMC cultures may furthermore comprise human serum from the PBMC donor.

The term “about” as used herein shall have the meaning of within 10%, more preferably within 5%, of a given value or range. In particular, in some embodiments, the cell population of the invention is introduced and/or cultured to have in the culture device a density of about 100, i.e. from about 90 to about 110, cells per mm² growth area to about 30000, i.e. about 27000 to about 33000, cells per mm² growth area. More preferably, the cell population is incubated at a density of about 500, i.e. about 450 to about 550, cells per mm² growth area to about 20000, i.e. about 18000 to about 22000, cells per mm² growth area, about 1000, i.e. about 900 to about 1100, cells per mm² growth area to about 10000, i.e. about 9000 to about 11000, cells per mm² growth area, about 1000, i.e. about 900 to about 1100, cells per mm² growth area to about 5000, i.e. about 4500 to about 5500, cells per mm² growth area, or about 1000, i.e. about 900 to about 1100, cells per mm² growth area to about 3000, i.e. about 2700 to about 3300, cells per mm² growth area. Most preferably the cell population is incubated at a density of about 2000, i.e. about 1800 to about 2200, cells per mm² growth area.

The term “growth area” as used within the meaning of the invention refers to the surface within a culture device upon which cells rest. The “density” as used within the meaning of the invention is the quantity of cells per unit area of the surface within the device upon which the cells rest.

The culture device may be produced of any material compatible with cell culture, in particular, non-cytotoxic cell culture tested material. Examples for the material are plastic materials, e.g., thermoplastic or duroplastic materials. Examples of suitable plastics are polyethylene, polypropylene, polysulfone, polycarbonate, polyetherethylketone (PEEK) or polytetrafluorethylene (PTFE). Typical culture devices known in the art and of use in the invention include culture flasks, dishes, plates, and multi-well plates.

In a further embodiment, the invention relates to a method wherein the cell population is obtained from a primary cell culture, a cell line or a cell strain, in particular from a primary patient sample or a combination thereof. Moreover, the invention also relates to methods wherein the cell population is obtained from a cell donor. Accordingly, the methods of the present invention are not particularly limited in terms of the cell populations used. In a particularly preferred embodiment of the methods of the invention the cell population comprises non-adherent cells or a mixture of non-adherent and adherent cell lines.

The term “primary culture”, as used herein, refers to the stage of the cell culture after the cells have been isolated from a cell donor and proliferated until they occupy almost all of the available substrate (i.e., reach 70% confluence or higher), under conditions well known to those skilled in the art. Cells may subsequently be subcultured (i.e., passaged) by transferring them to a new vessel with fresh medium to provide more room for continued growth.

The term “cell line”, as used herein, refers to the first subculture after the primary culture, and is also commonly referred to as the “subclone”. Typically, cell lines derived from primary cultures have a limited life span (i.e., they are finite), and as they are passaged, cells with the highest growth capacity predominate, resulting in a degree of genotypic and phenotypic uniformity in the population.

The term “cell strain”, as used herein, refers to a subpopulation of a cell line that is positively selected from the culture by cloning or other methods known to those skilled in the art, this cell line becomes a cell strain. A cell strain often acquires additional genetic changes subsequent to the initiation of the parent line.

In the case of non-adherent cells that are to be used, for example PBMCs or bone marrow cells, it is known that such cells typically do not form strong contacts with cell-culture surfaces or strong cell-to-cell contacts. Therefore, the cell monolayers used in the present invention, in particular the PBMC monolayers used in various aspects of the present invention are not envisioned to be necessarily equivalent to monolayers of adherent cells as understood in the art, i.e., comprising a layer of cells firmly attached, evenly spread, and covering the majority of the culture surface. Rather, in some embodiments, the cell monolayer, in particular the PBMC monolayer used in the invention may comprise cultures of high density comprising a majority of cells in direct contact with one or more other cells, but not necessarily adhered to the culture surface, or may comprise cultures of low density, wherein cells are within the monolayer but have no (direct physical) contact with any other cell in the culture. The cell monolayers used in certain aspects of the present invention may also comprise cultures of intermediate density, having discrete areas wherein cells are in contact with one or more cells and other areas where the cells exhibit no contact with other cells.

The cells, such as PBMCs, bone marrow cells, or other adherent-and non-adherent primary cells for use in the methods of the present invention may be isolated from a sample obtained from a healthy subject, i.e. not suspected to suffer from a disease or suspected to be predisposed to a disease, or may be isolated from a sample obtained from a subject known to be suffering from a disease or suspected to suffer from a disease. The diagnosis of the disease state of the subject may be made by standard methods routinely performed by those skilled in the art, e.g., physicians.

In another embodiment, the invention relates to methods wherein air-drying immobilizes the cells onto a surface. As described hereinabove, the method of the present invention immobilizes the cells onto a surface in a gentle and non-disruptive manner such that the cell population retains its in vivo state. The surface may be any surface of a culture device as described hereinabove.

In a further specific embodiment, the invention relates to methods wherein the sample evaporator supplies pressurized air at an air speed of 0.5 to 2.5 liters per minute per mm² (L/min/mm²), preferably 1.0 to 2.0 L/min/mm², more preferably 1.5 L/min/mm². The sample evaporator of the present invention is not particularly limited, however, in preferred embodiments of the method, the sample evaporator comprises a needle or a plurality of needles that supplies pressurized air to the cell population. It will be understood that the air speed, i.e. the velocity of air supplied, may need to be optimized depending on the diameter of the vessel supplying the pressurized air and the distance of the vessel from the surface of the liquid medium comprising the cell population.

As is demonstrated in the appended Examples, an optimal air speed could be determined, particularly for lymphocytes which are smaller in size and are less likely to be attached. Low air speeds, i.e., less than 1.5 L/min/mm², require longer drying times, and therefore may result in cell loss over time, high air speeds, i.e., more than 2.5 L/min/mm² may result in the formation of holes in the monolayer. The air speeds of the methods of the present invention may therefore need to be optimized for various cell populations, however, as can be appreciated by a person skilled in the art, no undue experimentation is necessary for a skilled person to identify optimal conditions for a specific cell population.

In a further embodiment, the invention relates to methods wherein air-drying is continued until the cell population is dry and the cell monolayer is created, preferably wherein the cell population is air-dried between 10 min and 12 hrs, more preferably between 10 min and 6 hrs.

As can be understood by a person skilled in the art, the duration of the air-drying method is dependent on, inter alia, the type of cells comprising the cell population or cell monolayer, the amount of liquid media, the air speed or velocity at which air is supplied by the sample evaporator and the distance between the sample evaporator supplying air and the surface of the liquid media. However, as will be understood by a person skilled in the art, the duration of air-drying should be kept to a minimum by optimizing the air speed and amount of liquid media in order to avoid cell loss due to degradation over time.

In a further embodiment, the invention relates to a method wherein the temperature of the supplied air is between 10 to 50 °C, preferably between 20 to 40 °C, more preferably between 23 to 37 °C. In a preferred embodiment, the invention relates to a method wherein the temperature of the supplied air is between 23 to 25 °C.

For practical purposes the methods of the present invention may use pressurized air supplied at ambient temperature, i.e. 23 to 25 °C, thus reducing the need for specialized equipment to heat the air supplied to the cell population and/or cell monolayer. However, a person skilled in the art will also recognize that air-drying of the cell population may be accelerated by use of a heated air supply, that is air supplied at a temperature above 25 °C. Moreover, the air-drying method may use air that is supplied at a temperature below ambient temperature, i.e. below 23 °C, for cell populations which prefer temperatures below 23 °C Therefore, the methods of the invention may also use an air-cooling system in conjunction with the sample evaporator to provide air that is below 23 °C.

In a specific embodiment, the invention relates to methods wherein prior to air-drying the cell population, part of the liquid media is removed, preferably wherein the liquid media is removed until at least 1 mm of liquid media remains above the surface of the cell population.

As described hereinabove, the inventors found that degradation of the monolayer, i.e. degradation due to, inter alia, cell loss or changes in cell morphology or cell-cell interactions, may occur if air-drying is prolonged, for example for more than 11 hrs for a cell population comprising PBMCs. As will be understood by a person skilled in the art, the duration of air-drying may be reduced by minimizing the amount of liquid media that needs to be evaporated, therefore in some embodiments of the methods of the invention part of the liquid media may be removed, by for example automatic or manual aspirators such as automatic or manual pipettes. The inventors found that, in some embodiments of the methods of the invention, it is preferred that the liquid media is removed until at least 1 mm, preferably between 1 mm and 5 mm, between 1 mm and 4 mm, between 1 mm and 3 mm, between 1 mm and 2 mm, of liquid media remains above the surface of the cell population. By removing the liquid media, the duration of the air-drying procedure may be reduced, enabling the high-throughput creation of the cell monolayers.

In a preferred embodiment, the invention is a method wherein the culturing uses a multi-well plate.

As described hereinabove the cell population of the present invention may be cultured or incubated in any culture device known to those skilled in the art. Of particular use are multi-well plates, which provide the ability to separately maintain multiple cultures, e.g., for multiple perturbations, with minimal material requirements, e.g., minimal media requirements. Preferred culture devices include 96 well plates, 384 well plates and 1536 well plates, most preferred are 384 well plates. As known in the art in connection with imaging analysis of cultures, in particular, fluorescence imaging, it is particularly preferred to use black wall plates specifically designed for imaging that reduce background fluorescence/background optical interference with minimal light scatter and reduced crosstalk. The culture device may be sterilized. In a most preferred embodiment, a multi-well imaging plate is used, the plate including multiple wells, wherein at least some of the wells comprise a first chamber, the first chamber being formed by one or more first sidewalls and a bottom wall; a second chamber, the second chamber being formed by one or more second sidewalls and including an opening for introducing liquids, wherein the second chamber is arranged on top of the first chamber; an intermediate floor provided between the first chamber and the second chamber which forms a disturbance blocking structure; wherein the intermediate floor is provided with at least one through hole that provides a liquid connection between the first and second chambers; wherein the through hole is configured for a tip of a pipette being inserted through the second chamber into the first chamber through said through hole.

The device is in particular of use in automated imaging systems and analysis. Thus, it is preferred that the device/culture device is suitable for use in such systems. In a non-limiting example, the culture device may be translucent. Culture dishes and plates of use for imaging, are well known in the art and are commercially available. A non-limiting example of a commercially available culture plate for use in the practice of the invention is Corning® 384-well, tissue-culture treated black lid, clear bottom plates (Corning Inc., Massachusetts, USA) or Corning® 384 Well Flat Clear Bottom Black Polystyrene TC-Treated Microplates (Product #3712). Another example is the Perkin Elmer PhenoPlate®.

Within the present invention, due to gravitational forces, cells settle at the bottom of a culture vessel without stacking atop one another. The air-drying process further improves that cells do not settle on top of each other, resulting in no visible cell clumping at the specified densities. As provided herein, the monolayers of the invention prepared by the methods of the invention, after undergoing air-drying and optionally fixation, for example methanol fixation, exhibit improved stability.

In one embodiment, the invention relates to a method wherein the method further comprises a step of chemically fixing the cell monolayer.

In order to increase stability of the cell monolayer subsequent to culture for further analysis, the practice of the invention may comprise a step of fixing the cells where the monolayers are stained and/or subsequently imaged. Fixing can be done by means and methods well-known to a person skilled in the art. For example, the cell monolayer can be fixed on the bottom area of the culturing device using methanol, formaldehyde or other known fixatives. Fixing is normally performed immediately prior to the addition of the means for visualizing the cells, cell components, and/or cellular proteins. If desired, a detergent can be added for cell permeabilization. An exemplary detergent is Triton X-114 for permeabilization. Because the invention, in part, relies on a cell monolayer, fixation and/or permeabilization must be implemented so as not to destroy the monolayer. In this respect, the practices described for removing and/or replacing medium, such as air-drying or careful use of an automatic or manual aspirator, can also be implemented for use of fixatives and/or permeabilizers. As the skilled person appreciates, subsequent to fixation, the monolayer will be more robust, i.e., resistant to disruption, e.g., from liquid forces. The methods of the invention may comprise the further step of fixing the cells prior to or subsequent to the step of air-drying, in preferred embodiments, the method comprises the further step of fixing the cells subsequent to the step air-drying.

The methods of the invention may further comprise a step of adding a dye prior to fixation, e.g. a viability dye. This may be done in order to verify that the cells in the cell monolayer are viable. Adding a dye, e.g. a viability dye, may be done by e.g. partially removing the supernatant and adding a dye, e.g. a viability dye, known to be suitable for visualizing the viability of cells, such as PBMCs. In particular, a viability dye may be added which can distinguish between live and dead cells to determine the viability of cells prior to the fixation and/or permeabilization required for the optional intracellular antibody staining or prior to elimination of biohazardous materials using formaldehyde fixation. Viability stain is based on the dynamic incorporation of the dye as a labeling agent into the cell membrane or cell organelles, or conversion of a dye precursor by cell enzymes or by detecting intermediates of the respiratory chain, or by intercalation in DNA or RNA. A person skilled in the art is well aware that dyes suitable for visualizing fixed cells may also be used. Suitable dyes and their application protocols are known to the skilled person and documented, for example, in the Molecular Probes Handbook, A Guide to Fluorescent Probes and Labeling Technologies. As a non-limiting example, the viability dye may be added in a 1:1000 mix dissolved in isotonic solution, e.g. PBS. In this regard, Invitrogen live/dead fixable 488 dye is particularly useful.

The practice of the invention may also comprise the addition of a detectable label to the cell monolayers (either in connection with label-free methods or independently), which label may be detected using microscopic methods. The detectable labels may label discrete cellular structures, components or proteins as known in the art. The label may also be attached to antibodies to specifically label and allow the detection of the antibody antigen. In a preferred embodiment, the detectable label allows visualization of the label under visible or ultra-violet light. Thus, the detectable label may be fluorescent. A multitude of visual labels are known in the art and are suitable for the invention. The labels may be detectable without further action or may only become detectable after performance of a secondary step, e.g., addition of a substrate, exposure to enzymatic reactions, or exposure to specific light wavelengths.

Cellular subpopulations (target cells) may be identified by detectable labels via expression of one or more markers on the surface of the target cell or inside of the cell. Alternatively, or additionally, subpopulations of cells may be defined by the lack of expression of one or more markers on the surface of the target cell or inside the target cell. It may be desirable to test for expression or lack of expression of one or more markers (e.g., two markers, three markers, four markers, etc.) to provide further assurance that a cell expressing or not expressing a marker is in fact a target cell, e.g., a member of desired subclass of PBMC cell. For example, a “cocktail” of antibodies to different markers may be each coupled (whether directly or indirectly) to the same label or to different labels. As an example, a cocktail of antibodies to different markers may each contain a binding motif that binds the same label (e.g., each may contain an Fc of the same species that is recognized by the same secondary antibody, or each may be biotinylated and specifically bound by the same avidin-coupled label). Optionally, two or more different antibodies or cocktails of antibodies may be utilized. Preferably the cells are stained using at least two labels that can be distinguished from one another, thereby permitting identification of cells that express at least two different markers of the target cell types. Cells may also be stained using at least three, four, five, or more different labels that can be distinguished from one another, thereby permitting detection of cells that express greater numbers of markers of the target cell type. Optionally, a cell may be identified as a cell of the target type if it expresses a preselected number of markers or certain preselected combinations of markers or a cell may be identified as a cell of the target type if it does not express a preselected marker. Additionally, it is not necessary that the marker(s) of the target cell type be unique to the target cells, as long as they permit distinction of the target cells from other cells in the population. Major PBMC cell populations are represented by CD11C for dendritic cells, CD14 for macrophages, CD3 (CD4 or CD8 with CD3) for T-cells and CD19 for B-cells. While the foregoing markers overlap on subsets of these major classes of PBMCs, staining with these markers for identifying subpopulations of PBMCs is widely accepted in the field. Further markers suitable for use in methods of the present disclosure may be found in the CD marker handbook (Becton, Dickinson and Co. 2010, CA, USA).

It is possible to use antibodies conjugated to detectable labels in the practice of the invention. Such antibodies allow the targeting of discrete cellular structures and, thus, cocktails of such antibodies (each bearing a different label) may be used to simultaneously visualize multiple targets/cellular structures/cellular components. Again because the invention relies on a cell monolayer, care must be taken during staining to avoid monolayer disruption. As the skilled person appreciates, this is particularly problematic with the use of antibody-based labels, as their use normally requires one or more wash-steps to eliminate unbound label that would interfere with accurate visualization, i.e., would result in non-specific staining and/or assay “noise”. Accordingly, the invention encompasses methods for the staining of PBMC monolayers with a detectable label, in particular, an antibody-based label, which minimizes or eliminates washing requirements subsequent to staining. The methods of the invention may comprise adding the detectable label(s) at concentrations that avoid generation of noise signal in the absence of washing, which can be determined by methods well known in the art and/or described herein. Thus, the invention encompasses the use of labeled antibodies at concentrations above or below that recommended by the antibody manufacturers.

For some exemplary cell types, cells may only be considered positive for a given marker if that marker exhibits a characteristic localization or pattern within the cell. For instance, a cell may be considered “positive” if a cytoskeletal marker is present in the cytoskeleton and “negative” if there is some diffuse cytoplasmic staining. In such a case, cells may be cultured under suitable conditions (e.g., as adherent cultures) to establish the characteristic localization or pattern within the cell. Suitable culture conditions and time for cytoskeleton assembly (or other processes to establish subcellular organization) that may be necessary for robust detection of a given marker are readily determined by those of ordinary skill in the art. Additionally, markers may readily be chosen which decrease or eliminate the need for adherent culture as a precondition to robust staining.

Dyes useful in labeling proteins are known in the art. In general, a dye is a molecule, compound, or substance that can provide an optically detectable signal, such as a colorimetric, luminescent, bioluminescent, chemiluminescent, phosphorescent, or fluorescent signal. In a preferred embodiment of the invention, the dye is a fluorescent dye. Non-limiting examples of dyes, some of which are commercially available, include CF dyes (Biotium, Inc.), Alexa Fluor dyes (Invitrogen), DyLight dyes (Thermo Fisher), Cy dyes (GE Healthscience), IRDyes (Li-Cor Biosciences, Inc.), and HiLyte dyes (Anaspec, Inc.). In some embodiments, the excitation and/or emission wavelengths of the dye are between 350 nm to 900 nm, or between 400 nm to 700 nm, or between 450-650 nm.

In another embodiment, the invention relates to a method wherein the method further comprises, subsequent to step (c), a step (d) comprising staining the monolayer.

Cell staining is a technique that can be used to better visualize cells and cell components in cellular imaging and microscopy. As is understood by one skilled in the art different stains may be used to preferentially stain certain cell components, such as a nucleus or a cell membrane, or the entire cell. Most stains can be used on fixed, or non-living cells, while only some can be used on living cells; some stains can be used on both living or non-living cells. Within the methods of the present invention, the stain is typically added after air-drying the cell monolayer, however methods wherein the stain is added before air-drying are not excluded. The pH of the liquid media may, in some instances, influence the effect of the stain, and thus may be removed, preferably by air-drying prior to staining the cell monolayer.

Staining may, for example, comprise using multiple detectable labels, e.g., antibodies, self-antibodies or patient serum. A stain may be observable under visible light and under ultraviolet light. A stain may comprise an antibody directly or indirectly coupled to a colored reagent or an enzyme capable of producing a colored reagent. When antibodies are used as a component of a stain, a marker can be directly or indirectly coupled to the antibody. Examples of indirect coupling include avidin/biotin coupling, coupling via a secondary antibody, and combinations thereof. For example, cells may be stained with a primary antibody that binds a target-specific antigen, and a secondary antibody that binds the primary antibody or a molecule coupled to the primary antibody can be coupled to a detectable marker. Use of indirect coupling can improve signal to noise ratio, for example by reducing background binding and/or providing signal amplification.

The stain may also comprise a primary or secondary antibody directly or indirectly coupled (as explained above) to a fluorescent label. The fluorescent label may be selected from the group consisting of: Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750 and Alexa Fluor 790, fluoroscein isothiocyanate (FITC), Texas Red, SYBR Green, DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine, aminoacridine, and quantum dots.

Further exemplary embodiments of the present method utilize antibodies directly or indirectly coupled to a fluorescent molecule, such as ethidium bromide, SYBR Green, fluorescein isothiocyanate (FITC), DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N, N-diethyl-4-(5′-azobenzotriazolyl)-phenylamine and aminoacridine. Other exemplary fluorescent molecules include quantum dots, which are described in the patent literature [see, for example, U.S. Pat. Nos. 6,207,299, 6,322,901, 6,576,291, 6,649,138 (surface modification methods in which mixed hydrophobic/hydrophilic polymer transfer agents are bound to the surface of the quantum dots), U.S. Pat. Nos. 6,682,596, 6,815,064 (for alloyed or mixed shells), each of which patents is incorporated by reference herein)], and in the technical literature [such as “Alternative Routes toward High Quality CdSe Nanocrystals,” (Qu et al., Nano Lett., 1(6): 333-337 (2001)]. Quantum dots having various surface chemistries and fluorescence characteristics are commercially available from Invitrogen Corporation, Eugene, Oreg., Evident Technologies (Troy, N.Y.), and Quantum Dot Corporation (Hayward, Calif.), amongst others. Quantum dot” also includes alloyed quantum dots, such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN. Alloyed quantum dots and methods for making the same are disclosed, for example, in US Application Publication No. 2005/0012182 and PCT Publication WO 2005/001889.

In a preferred embodiment, the invention relates to methods wherein staining involves staining with a histological stain.

As described hereinabove, the methods of the present invention may use any stain known to those skilled in the art, such as fluorescent and/or immunohistochemistry stains, for the visualization of the cell monolayer. However, as described hereinabove, fluorescent and/or immunohistochemistry stains may not be well suited for the detection/visualization of cell populations obtained from a donor, in particular in a clinical setting, due to, inter alia, the need for expensive equipment and material, such as fluorescence microscopes and antibody-based labels, the time-consuming nature of the staining and visualization methods and the need to customize stains for specific diseases and at times patients. Nevertheless, methods involving the use of fluorescent and/or immunohistochemical stains are not excluded from the methods of the present invention, in particular fluorescent staining methods, as these may provide advantages such as high-resolution images with high specificity.

The inventors of the present method surprisingly found that conventional histological stains may be used for cell visualization, cell detection and/or cell identity analysis. The histological stains utilized in the present method may be any histological stain known to those skilled in the art, however it is preferred that the histological stain is one commonly used in clinical pathology/histology. The use of histological stains in the present method provides several advantages over the stains currently employed in the state-of-the-art. Histological stains are significantly cheaper than fluorescent and immunohistochemistry stains and do not require extensive equipment and/or materials for their use and/or subsequent visualization. A further advantage is that histological stains may be used for all cells since they do not depend on the expression of selected proteins, as is the case for immunochemical stains, therefore the histological stains may be applied for multiple samples, such as those derived from multiple cancer indications, thus increasing useability and convenience in a clinical setting. Moreover, the use of the same staining protocol for all cell samples using the histological stain(s) of the present invention provides a major advantage over the use of immunohistochemistry stains as these require staining panels to be adapted for each disease indication. A further advantage of the methods of the invention is all staining and fixation steps may be fully automated, thus providing a robust and high-throughput platform for cellular analysis. In methods currently known in the art, high-throughput techniques were less feasible using immunohistochemistry stains and consumables due to the high cost of said stains and consumables and automated liquid-handling equipment, such as automated or robotic aspirators, which require significantly larger quantities of materials. Finally, histochemistry stains are compatible with the use of auto-fluorescent therapeutics, which cannot be assayed with the existing technology based on fluorescent staining.

Numerous methods of sample preparation and histological staining are known in the art and commonly performed, e.g., to aid in research or diagnosis. See, e.g., O'Leary (ed.), 2003, Advanced diagnostic methods in pathology: principles, practice, and protocols, W. B. Saunders, Philadelphia; and Kiernan, 2002, Histological and histochemical methods: theory and practice, Arnold, London.

Specific staining techniques useful in the methods described herein include hematoxylin and eosin (H&E) stain, hematoxylin stain, eosin stain, Giemsa stain, Pappenheim stain, May-Gruenwald stain, methylene blue stain, azure B stain, Masson triple or trichrome stain, aldehyde fuchsin stain, Verhoeff-van Gieson elastic tissue stain, silver stain, Periodic acid Schiff stain, acid fast stain, alcian blue stain, alcian blue-PAS stain (PAB), hyaluronidase digestion for alcian blue alizarin stain for calcium, auramine-rhodamine stain, Bielschowsky stain, bile stain, Bodian's stain, colloidal iron stain, congo red stain, copper stain, elastic stain, elastic van Gieson stain, elastic—Weigert's resorcin-fuchsin method, modified elastic van Gieson stain, Fontana-Masson stain for melanin, melanin bleach, Fraser-Lendrum stain, Giemsa (modified May-Gruenwald) stain, Gram stain, Gridley's stain, Grimelius argyrophil Stain (Pascual's Method), Grocott's methenamine silver (GMS) Stain, Holzer's glial fiber stain, Hortega's pineal stain, iron stain (Prussian blue), iron stain (Turnbull's blue), Jones' silver stain, Leishman/Giemsa stain, Luxol Fast Blue (LFB) stain, Luxol Fast Blue (LFB) and cresyl violet, Methyl Green Pyronin (MGP) stain, mucicarmine stain, Nissl stain, oil red O stain, Orcein stain, osmium tetroxide, Papanicolaou stain, Periodic acid-Schiff stain (PAS), digested stain (PAS-D), phosphotungstic acid-hematoxilin (PTAH) stain, reticulin stain, Schmorl's stain, silver stain for reticulin, spirochete stain (Steiner & Steiner method), Sudan Black B stain, trichrome stain—Masson's method, trichrome Stain—microwave method, thioflavin S stain, modified thioflavin S stain, toluidine blue stain, Terminal dUTP nick end labeling (TUNEL) assay, urate crystal stain, VonKossa stain for calcium or Wright stain. Chromophoric histochemicals may also be used. Chromophoric histochemicals are compounds that create pigmented material through chemical reactions between the chemicals and components within a sample or exogenously added to the sample. A commonly performed histochemical technique is the Perls Prussian Blue reaction, used to demonstrate iron deposits in diseases like hemochromatosis.

In a specific embodiment, the invention relates to method wherein the histological stain is a hematoxylin and eosin stain (H & E stain), hematoxylin stain, eosin stain, Giemsa stain, Pappenheim stain, May-Gruenwald stain, methylene blue stain, azure B stain, Masson triple or trichrome stain, aldehyde fuchsin stain, Verhoeff-van Gieson elastic tissue stain, silver stain, Periodic acid-Schiff stain, Gram stain, Papanicolau's (Pap) stain, or Immunoperoxidase stain, or a combination thereof.

As outlined in “Blood Cells-A Practical Guide, Fifth Edition” by Barbara J. Bain, it is crucial to ensure cells are thoroughly dried before staining with the above-mentioned stains, in particular histological stains, to attain optimal results. Without effective drying of the cells whilst they are still in an in vivo state and/or are morphologically intact state, the application of histological stains may, for example, not be effective when applied. The presented methods of the invention, involving air-drying with a sample evaporator is therefore essential to obtain optimal staining results with, e.g., histological stains, in particular when using multi well plates. The methods of the invention therefore enable high-throughput screening and imaging that is amenable to clinical settings, which has so far not been demonstrated in the prior art. The hereinabove mentioned histological stains are well known in the art and their appropriate use for the analysis of a cell sample is readily determined by one skilled in the art from the common general knowledge in the field. For example, hematoxylin and eosin stain (H & E stain) is one the most widely used stains in the clinical setting. The hematoxylin component principally colors the nuclei of cells blue, dark-purple or brown, along with a few other tissues, such as keratohyalin granules and calcified material. Eosin stains the cytoplasm and some other structures including extracellular matrix such as collagen and cell membranes in up to five shades of pink. The eosinophilic (i.e., substances that are stained by eosin) structures are generally composed of intracellular or extracellular proteins. The Lewy bodies and Mallory bodies are examples of eosinophilic structures. Most of the cytoplasm is eosinophilic and is rendered pink. Red blood cells are stained intensely red. This allows a pathologist to easily differentiate between the nuclear and cytoplasmic parts of a cell, and additionally, the overall patterns of coloration from the stain show the general layout, distribution and morphology of the cells and provides an overview of the cell population structure. Thus, pattern and morphology recognition, both by the human eye and by computer-implemented techniques provides histologic information on the cell sample. The results from H&E staining are not overly dependent on the chemical used to fix the tissue or slight inconsistencies in laboratory protocol, which contributes to its routine use in histology. A further example is the Giemsa stain, which is a nucleic acid stain, typically used in cytogenetics and for the histopathological diagnosis of an infectious disease. As is well known in the art, Giemsa stains are often used to stain peripheral blood and bone marrow cell samples, where erythrocytes stain pink, platelets show a light pale pink, lymphocyte cytoplasm stains sky blue, monocyte cytoplasm stains pale blue, and leukocyte nuclear chromatin stains magenta. Giemsa stain is also used to visualize chromosomes, such as a method known as Giemsa banding (commonly called G-banding) to stain chromosomes and often used to create a karyogram (referred to as a chromosome map). It can identify chromosomal aberrations such as translocations and rearrangements. Other common uses of the Giemsa stain are to visualize Yersinia pestis, Trichomonas vaginalis, Histoplasma, Chlamydia and Plasmodium species. Giemsa stain may be combined with Wright stain to form Wright-Giemsa stain, which can be used to study the adherence of pathogenic bacteria to human cells and differentially stains human and bacterial cells purple and pink respectively.

Within the methods of the invention, a combination of stains (commonly referred to as differential staining), such as those described hereinabove may be used on the same cell population to differentially stain various cellular components and microorganisms. For example, differential staining is used to detect abnormalities in the proportion of different white blood cells (WBCs) in the blood. The process or results are called a WBC differential. This test is useful because many diseases alter the proportion of certain white blood cells. By analyzing these differences, it may be possible to diagnose disease. The skilled person is able to readily determine an appropriate combination of stains for the analysis of a cell population.

In some embodiments of the methods of the invention, the staining involves staining with a fluorescent stain.

Fluorescent stains may be used to stain, for example, DNA, cell membranes, cell surface, mitochondria, lysosomes, lipid droplets, cytoskeleton, vesicles, cytoplasm. Fluorescent stains include, but are not limited to, the stains mentioned herein above including stains comprising fluorescent labels. These stains are typically commercially available and well known to the person skilled in the art. An exemplary method for the use of fluorescent stains is depicted in Example 8 and FIG. 9, where fluorescent antibodies targeting the T-cell surface proteins CD4, CD8 and CD3 and a nuclear dye, DAPI, was used.

The enhanced stability of the monolayer, as demonstrated in this invention, surpasses that of previous methodologies, facilitating the implementation of cyclic staining procedures and multiplexed staining techniques of non-adherent cells. This advancement permits repeated applications of various stains without compromising the integrity of the cellular structure, thereby allowing for a more comprehensive and intricate analysis of cellular components. This capability is especially beneficial in complex biological studies where simultaneous visualization of multiple targets, such as different cell surface proteins and nuclear components, is required. The robustness of the monolayer in this approach ensures that it remains intact and responsive to successive rounds of staining, offering an improved tool for detailed cellular analysis in research and diagnostic applications.

In further embodiments of the methods of the invention, subsequent to staining the monolayer with a histological stain, the method further comprises a step of staining the monolayer with a fluorescent stain.

The integration of fluorescent dyes alongside traditional histological stains introduces a novel approach for concurrent molecular and morphological analysis of cells. This dual-staining technique, as demonstrated in Example 9 and FIG. 10, allows for the precise correlation of specific molecular markers with cellular morphology. Such synergy between molecular readouts and morphological quantification significantly enhances the accuracy and depth of cellular analysis. By enabling the simultaneous observation of molecular expressions and structural details within matched same cells, the method provides a more comprehensive understanding of cellular behaviors and characteristics.

In some embodiments, the invention relates to a method for determining whether a cell donor suffers from a disease using the monolayer created by the methods of the invention, the method comprising: a) providing the cell monolayer created by the methods of the invention; b) analyzing the cell monolayer; and c) determining whether the cell donor suffers from a disease.

In particular embodiments, the methods of the present invention involves analyzing the cell monolayer. Said analysis comprises the analysis of cell viability, cell morphology, cell-cell interactions, membrane integrity, the identification or measurement of a biomarker, the presence of a pathogen or a combination thereof. Preferably, cell morphology is used for the analysis of the cell monolayer.

The inventors of the present invention found that the analysis of cell morphology is particularly advantageous in the identification and characterization of the cell monolayer to determine whether a cell donor suffers from a disease or whether the donor's diseased cells will respond to a treatment, as diseased cells, in particular tumor cells, have a distinct morphology. Tumor cells are typically morphologically characterized by a large nucleus which may have an irregular size and shape, the nucleoli are typically prominent, and the cytoplasm may be scarce and intensely colored or pale.

The term “cell morphology”, as used herein, refers generally to the form, structure and configuration of a cell and may include aspects of the cell appearance, such as the shape, color, texture or pattern or internal or external elements of the cell(s). The terms “form” or “shape” of the cell, as used herein, refers to the typical cell forms such as circular cells, elliptic cells, shmoo like cells, division forms like dumbbells, star-like cell forms, flat cells, scale-like cells, columnar cells, invaginated cells, cells with concavely formed walls, cells with convexly formed walls, the presence of prolongations, appendices or cilia, the presence of angles or corner etc. Typical morphologies or forms would be known to the person skilled in the art and can be derived from Junqueira et al., 2002, Basic Histology, Mcgraw-Hill editors. The term “cell size”, as used herein, is to be understood as the physical dimensions of the cell, in particular the surface area of the cell.

The methods disclosed herein which provide a cell monolayer may be isolated from a sample obtained from a healthy subject, e.g., not suspected to suffer from a disease or suspected to be predisposed to a disease or may be isolated from a sample obtained from a subject known to be suffering from a disease or suspected to suffer from a disease. The diagnosis of the disease state of the subject may be made by standard methods routinely performed by those skilled in the art, e.g., physicians or using the methods of the present invention. Moreover, when a sample is obtained from a subject suspected to be predisposed to a disease or from a healthy subject, a prognostic evaluation may be provided by using standard methods performed by those skilled in the art or by the methods provided by the present invention.

The term “diagnosis” (along with grammatical variations thereof such as “diagnosing” or “diagnostic”) refers to the identification of a molecular or pathological state, disease or condition, such as the identification of cancer, or refers to the identification of a cancer patient who may benefit from a particular treatment regimen.

The term “prognosis” (and grammatical variations thereof such as “prognosing” or “prognostic”) refers to the prediction of the likelihood of benefit from a treatment such as a cancer therapy.

The term “prediction” or “predicting” is used herein to refer to the likelihood that a patient will respond either favourably or unfavourably to a particular therapeutic agent. In one embodiment, prediction or predicting relates to the extent of those responses. In one embodiment, the prediction or predicting relates to whether and/or the probability that a patient will survive or improve following treatment, for example treatment with a particular therapeutic agent, and for a certain period of time without disease progression.

It is one surprising advantage of the disclosed cell monolayer and methods using the same, that both healthy and diseased cells may be comprised in the monolayer. “Diseased cells” within the meaning of the invention relate to PBMCs being affected by a disease and thus distinguishable from healthy cells. In particular, in some embodiments, a diseased cell will show differential expression of marker molecules that enable their specific detection using the methods of the present invention. For example, a diseased PBMC may show expression of known cancer markers, in particular markers for lymphoma or leukemia, which enable their detection and discrimination from healthy PBMCs.

In some embodiments, in addition to healthy and diseased cells, both adherent and non-adherent cells are present in the monolayer of the invention. Therefore, the methods of the invention may provide unique advantages over methods known in the art, by allowing to obtain a total overview of the current status with regard to the presence or absence of subpopulations of cells and/or the distribution of different subpopulations in the sample of the cell donor. In contrast, methods known in the art rely on prior isolation of subpopulations thereby neglecting the information contained in cell-cell interactions between different subpopulations. The cell monolayer of the invention and as, e.g., produced by the methods of the invention, can thus be used in some embodiments for determining whether the cell donor suffers from a disease or has a predisposition for a disease by adding a detectable label or stain to the cell monolayer which is specific for a cell type indicative for the presence of a disease or by determining altered ratios among the various subpopulations of the cells, which ratios are indicative of the disease or predisposition for the disease. Accordingly, the present invention also provides, in some aspects, for a method for diagnosing a disease or predisposition to a disease in a cell donor comprising the cells cultured according to any of the methods of the invention. The cell monolayer of the invention and the methods of the invention may thus also be used for following the course of a disease during treatment of a disease or in the absence of treatment.

The method includes isolating cells from a subject previously or currently treated for an disease, stimulating the cells, identifying subpopulations of the cells, comparing data from the cell subpopulations to a subject response to the therapeutic and selecting a signature marker profile related to a positive response to the therapeutic, thereby monitoring the course of therapy.

The disease to be diagnosed using the methods of the invention is not particularly limited as long as it can be diagnosed using cell population obtained from the cell donor, i.e. a subject having the disease to be diagnosed shows an altered cellular pattern that can be associated with the disease, e.g., ratios of PBMC sub-populations that are altered from those expected in a healthy donor. Diseases that are in particular diagnosable and/or predictable by the methods of the invention include, but are not limited to, myeloproliferative disorders (or general blood cancers), inflammatory disorders, latent virus infections, cellular growth disorders, cellular chemotaxis disorders, metabolic disorders, autoimmune disorders (e.g., staining with self ligand or patient serum for clonal antibodies or self antigen recognition). Moreover, the methods of the invention can be used to diagnose leukemia (chronic and acute), lymphoma (mature B-cell, mature T-and NK cell, Hodgkin's lymphoma), HIV, gout, shock and the like.

In a preferred embodiment, the invention relates to a method wherein the disease is an infectious disease, solid cancer or liquid cancer.

In principle, any population of cells may be used in the methods of the present invention, and thus the methods of the present invention may be used to determine whether a cell donor suffers from an infectious disease, solid cancer or liquid cancer in preferred embodiments.

In some embodiments of the methods of the present invention, the infectious disease is caused by a pathogen, such as a bacterium, fungus, parasite or virus.

Said bacterium may be, but is not limited to, Borrelia, Staphylococcus, Streptococcus (e.g. Streptococcus pneumoniae), Nesisseria (e.g. Meningococcus), Clostridium, Yersinia (e.g. Yersinia pestis), Parabacteroides (e.g. Parabacteroides distasonis), Corynebacterium, Capnocytophaga, Pseudomonas, Legionella, Citrobacter (e.g. Citrobacter koseri), Escherichia (e.g. Escherichia coli), Klebsiella, Tropheryma (e.g. Tropheryma whipplei).The pathogenic fungus may be, but is not limited to, Candida (e.g. Candida tropicalis, Candida krusei), Pichia (formerly Hansenula anomala), Histoplasma (e.g. Histoplasma capsulatum), Cryptococcus (e.g. Cryptococcus neoformans), Talaromyces (e.g. Talaromyces marneffe formerly Penicillium marneffei), Malassezia (e.g. Malassezia furfur), Rhodotorula (e.g. Rhodotorula rubra). The pathogenic parasite may be, but is not limited to, single cell eukaryotes such as Plasmodium (e.g. Plasmodium vivax, Plasmodium falciparum, Plasmodium malariae, Plasmodium ovale, and Plasmodium knowlesi), Babesia (e.g. Babesia microti), Toxoplasma (e.g. Toxoplasma gondii), Trypanosoma or Leishmania and Nematoda such as Wuchereria bancrofti, Brugia malayi, Loa loa, Mansonella perstans, Mansonella ozzardi or Onchocerca volvulus. The pathogenic virus may be, but is not limited to, herpes simplex virus (HSV), cytomegalovirus (CMV), and human papillomavirus (HPV).

The methods of the present invention may be employed to directly or indirectly detect the presence of an infectious disease caused by a pathogen, such as a bacterium, fungus, parasite or virus. Direct detection refers to the direct identification of the pathogen in the cell sample, that is, the direct observation of the pathogenic species. For example, Borrelia, in particular Borrelia burgdorferi (the causative agent of Lyme disease) may be directly detected due to its unique morphology having an irregularly coiled spiral shape with a length between 10 and 40 μm and diameter between 0.2 and 0.3 μm. B. burgdorferi may be found in vitro in a non-uniformly coiled, twisted or intertwined state and is often found in aggregated form. The direct detection of B. burgdorferi may be facilitated by staining with histological stains, in particular with a May-Gruenwald stain, which has been demonstrated as an effective stain for this species. An example of a parasitic species which may be directly detected are various Plasmodium species which have distinct morphological features, for example, Plasmodium falciparum are characterized by the presence of young trophozoites (i.e., rings) and the absence of mature trophozoites and schizonts. The ring stages of P. falciparum tend to be slightly smaller than other Plasmodium species and are generally more numerous. Multiply infected red blood cells and appliqué forms are seen more often in P. falciparum than in other Plasmodium species. The crescent-shaped gametocytes of P. falciparum are very distinctive but tend to only appear late in the infection. Indirect detection of a pathogenic species refers to the detection of changes in the cellular characteristics in a cell sample which does not correspond to the cellular characteristics of the pathogen, these include cell viability, cell morphology, membrane integrity, the identification or measurement of a biomarker or a combination thereof. For example, a pathogen may be indirectly detected by observing morphological changes in white blood cells such as hypersegmentation or right shift of neutrophil nuclei, toxic granulation which is more basophilic and larger than in uninfected cells, formation of vacuoles, formation of Döle bodies, neutrophil aggregation and/or enlargement and the presence of phagocytosed organisms, parasites, platelets and red blood cells. For example, cells infected with the CMV typically appear enlarged with epithelial and endothelial cells having basophilic inclusions in both the cytoplasm and nucleus. HSV infected cells show clumping and margination of the chromatin, a ground glass appearance of the nuclei, and vacuolization. The indirect detection of viral infection may also include an evaluation of the cytopathic effect (CPE) of the virus. CPE refers to structural changes, e.g. morphological changes, in the host cell that are caused by viral infection. Common examples of CPE include rounding of the infected cell, vacuolization, swelling/clumping of host cell, fusion with adjacent cells to form syncytia and the appearance of nuclear or cytoplasmic inclusion bodies, which are accumulations of virus replication by-products, altered host cell organelles and/or structures. A person skilled in the art is well aware of methods of evaluating CPE in cell samples. Moreover, CPE may be used for diagnosis and drug screening, wherein a reduction in CPE acts as an indicator for the efficacy of a test compound/drug.

Solid cancers of relevance to the methods of the present invention include, but are not limited to, lung cancer (e.g., small cell lung cancer or non-small cell lung cancer), gastrointestinal cancer, colorectal cancer, colon cancer, anal cancer, liver cancer (e.g., hepatocellular carcinoma), pancreatic cancer, stomach cancer, genitourinary cancer, bladder cancer, biliary tract cancer, hepatobiliary cancer, testicular cancer, cervical cancer, ovarian cancer (e.g., cancerous ovarian teratoma), uterine cancer, endometrial cancer, vaginal cancer, vulvar cancer, malignant mesothelioma, esophageal cancer, laryngeal cancer, prostate cancer, breast cancer, brain cancer, neuroblastoma, Ewing's sarcoma, osteogenic sarcoma, kidney cancer, epidermoid cancer, skin cancer, melanoma, head and/or neck cancer, mouth cancer, thymoma, Merkel-cell cancer, and neuroendocrine cancer.

Moreover, the methods of the present invention may be applied to circulating tumor cells (CTCs), which are primary cells derived from solid tumors that have detached from the primary tumor and entered the peripheral blood circulation system or lymphatic system. CTCs may initiate tumor metastasis in sites other than the site of the primary tumor. CTCs may therefore be of use in the early diagnosis, prediction of prognosis, assessment of recurrent risk, supervision of curative effects and/or individualized treatment of patients. CTCs may also be cultured in vitro to build CTC lines for further studies on metastasis mechanisms, prevention, and intervention. It is however difficult to detect and analyze circulating tumor cells (CTCs) in the peripheral blood of metastatic cancer patients by conventional methods, such as flow cytometry, due to the fact that CTCs are estimated to occur at a frequency of approximately 1 CTC per 10⁶-10⁷ peripheral blood cells. Therefore, the detection and identification of CTCs is typically achieved through different techniques (such as, e.g., immunomagnetic separation, size-based filtration, microchip microfluidic capture, density gradient centrifugation, dielectrophoresis field flow fractionation, No-enrichment/ICC analysis, RT-qPCR analysis, CK19 mRNA-detection, protein assays and epispot) used in combination with CTC enrichment procedures. The methods of the present invention may also be applied to the detection and identification of CTCs in combinations with CTC enrichment procedures. CTC enrichment procedures are well known in the art, and a skilled person is able to determine the appropriate enrichment method for the sample. Non-limiting examples of CTC enrichment methods include enrichment methods based on physical properties (e.g., size, density, electric charge and deformability) such as microfiltration technologies, inertial focusing technologies and dielectrophoretic charge technology and enrichment methods based on biological properties (e.g. immunological procedures) such as procedures with antibodies against either tumor-associated antigens (positive selection) or common leukocytes antigen CD45 (negative selection).

The term “enrichment”, as used herein, refers to the process of substantially increasing the ratio of target bioentities (e.g., CTCs) to non-target materials in the processed analytical sample compared to the ration in the original biological sample. In cases where peripheral blood is used as the starting materials, red cells are not counted when assessing the extent of enrichment. CTCs may be enriched using a conventional technique as described hereinabove, thereby increasing the amount of detectable CTCs by 25%, 50%, 100%, 200%, 500% or more as compared to an unenriched sample.

Liquid cancer refers to hematopoietic cancers such as leukemia, lymphoma and myeloma. Such tumors include acute myelogenous leukemia (AML), acute lymphocytic leukemia (ALL), acute promyelocyte leukemia (APL), precursor and mature B cell neoplasms, chronic lymphocytic leukemia (CLL), plasma cell neoplasms, chronic myelocytic leukemia (CML), multiple myeloma, Hodgkin's lymphoma, non-Hodgkin's lymphoma, myelodysplastic syndromes (MDS), myelodysplastic and myeloproliferative diseases, chronic myelomonocytic leukemia (CMML), polycythemia vera, precursor and mature T cell neoplasms, T cell leukemias and lymphomas, mycosis fungoides, and Sezary syndrome. See, e.g., a summary of hematopoietic and lymphoid malignancies at Greer, et al. (eds.), WiNTROBE's CLINICAL HEMATOLOGY (Lippincott Williams & Wilkins 1 lth Ed. (2003)) at Table 71.3 (World Health Organization Classification of Hematopoietic and Lymphoid Neoplasms).

As already mentioned hereinabove, the methods of the invention may be applied to the detection of any disease, in particular diseases which may be identified and characterized by histological analysis. Therefore, the methods of the present invention may be used to determine whether a cell donor suffers from a disease other than an infectious disease or cancer. Such diseases include, but are not limited to, anemia, jaundice, sickle cell disease, thrombocytopenia, sudden kidney failure, G6PD deficiency and autoimmune diseases.

In preferred embodiments, the invention relates to methods wherein the solid or liquid cancer is a carcinoma, sarcoma, myeloma, leukemia, lymphoma or a mixed-type cancer.

In preferred embodiments of the methods of the invention PBMC or bone marrow cells are used for the creation of the cell monolayer. There are various diseases associated with cells comprised in a PBMC or bone marrow cell sample, in particular proliferative diseases such as cancer. Thus, in the methods of the present invention, in particular in the methods for determining whether a subject suffering from cancer will respond or is responsive to treatment with a test compound, the cancer is preferably a cancer associated with PBMCs or bone marrow cells or cells derived from PBMCs or bone marrow cells. The skilled person is aware of cancerous diseases falling within this definition, i.e. types of cancer associated with PBMCs or bone marrow cells or cells derived from PBMCs or bone marrow cells. However, the methods of the present invention are not limited to cancer. That is, the methods of the present invention can be used to determine whether a subject will respond/is responsive to treatment of the following diseases of the following ICD-10 codes (not limited thereto) A00-B99-certain infectious and parasitic diseases; C00 C97 malignant neoplasms; D70-77-other diseases of the blood forming system; D80-89-certain disorders involving the immune mechanism, not classified elsewhere; D82-Immunodeficiency associated with other major defects; D83-Common variable immunodeficiency; D84-Other immunodeficiency; G35-37-Diseases of the central nervous system; I00-I03-acute rheumatic fever; I05-I09-Chronic rheumatic heart disease; I01-Rheumatic fever with heart involvement; I06-Rheumatic aortic valve diseases; I09-Rheumatic myocarditis; I70-Atherosclerosis; K50-Crohn's disease; K51 Colitis; K52-other noninfective gastroenteritis and colitis; M00-M19 Athropathies; M05-Seropositive rheumatoid arthritis; M06-Other rheumatoid arthritis; M10-Gout; M11-Other crystal athropathies; M35-sicca syndrome; M32-Systemic lupus erythematosus; N70-77-inflammatory diseases of female pelvic organs; P35-39-infections specific to the perinatal period; P50-P61-hemorrhagic and hematological disorders of the fetus and newborn; Z22-Carrier of infectious disease; Z23-Need for immunization against single bacterial diseases; and/or Z24-Need for immunization against certain single viral diseases.

In a specific embodiment, the invention relates to methods wherein said cancer is glioblastoma (GBM) or acute myeloid leukemia (AML).

As is disclosed in the appended Examples, the methods of the present invention have been applied to both non-adherent cells and adherent cells, which are represented by AML and GBM cells respectively. Optimal therapeutic agents and regimens for AML and GBM could be identified using the methods of the invention, and is depicted in the appended Figures.

In one embodiment, the invention relates to a method for determining whether a cell donor suffering from a disease will respond or is responsive to treatment with a therapeutic agent comprising: a) providing a cell population; b) culturing said cell population in a liquid medium comprising the therapeutic agent; c) air-drying the cell population with a sample evaporator to create a cell monolayer; d) imaging the cell monolayer created in step (c); and e) assessing the response of the cells comprised in the cell monolayer to the therapeutic agent to determine whether the cell donor suffering from a disease will respond or is responsive to treatment with said therapeutic agent. “Response” or “responsive” refers to a cell monolayer or a subject showing at least one altered characteristic subsequent to treatment. The altered characteristic of the subject may be amelioration or slowing down of the targeted pathologic condition or disorder.

As used herein, the terms “prevent”, “preventing” and “prevention” refer to the prevention of the occurrence and/or recurrence or onset of one or more symptoms of a cancer disease in a subject resulting from the administration of a prophylactic or therapeutic agent. “Therapeutic agents” within the meaning of the invention are molecules including, without limitation, polypeptides, peptides, glycoproteins, nucleic acids, synthetic and natural drugs, peptoides, polyenes, macrocyles, glycosides, terpenes, terpenoids, aliphatic and aromatic compounds, and their derivatives. In a preferred embodiment, the therapeutic agent is a chemical compound such as a synthetic and natural drug. In another preferred embodiment, the therapeutic agent effects amelioration and/or cure of a disease, disorder, pathology, and/or the symptoms associated therewith. The polymers may encapsulate one or more therapeutic agents.

Suitable therapeutic agents include, without limitation, those presented in Goodman and Oilman's The Pharmacological Basis of Therapeutics (e.g., 9th Ed.) or The Merck Index (e.g., 12th Ed.). Genera of therapeutic agents include, without limitation, drugs that influence inflammatory responses, drugs that affect the composition of body fluids, drugs affecting electrolyte metabolism, chemotherapeutic agents (e.g., for hyperproliferative diseases, particularly cancer, for parasitic infections, and for microbial diseases), antineoplastic agents, immunosuppressive agents, drugs affecting the blood and blood-forming organs, hormones and hormone antagonists, vitamins and nutrients, vaccines, oligonucleotides and gene therapies. It will be understood that compositions comprising combinations, e.g. mixtures or blends of two or more active agents, such as two drugs, are also encompassed by the invention.

In one embodiment the therapeutic agent may be a drug or prodrug, antibody or vaccine. The method of the invention may be used to assess whether administration of a therapeutic agent to a patient triggers a response to the therapeutic agent, or a component of a delivery vehicle, excipient, carrier etc. administered with the therapeutic agent.

The precise nature of the therapeutic agent is not limiting to the invention. In non-limiting embodiments the method of the invention may be used to assess response to synthetic small molecules, naturally occurring substances, naturally occurring or synthetically produced biological agents, or any combination of two or more of the foregoing, optionally in combination with excipients, carriers or delivery vehicles. “Treatment” or “treating“ refers to both therapeutic treatment and prophylactic or preventative measures, wherein the object is to prevent, ameliorate or slow down (lessen) the targeted pathologic condition or disorder, or one or more symptom associated therewith. Similarly, “responsive to” or “responds” and analogous terms refer to indications that the targeted pathological condition, or one or more symptom associated thereof, is prevented, ameliorated or lessened. The terms are also used herein to denote delaying the onset of, inhibiting (e.g. reducing or arresting the growth of), alleviating the effects of, or prolonging the life of a patient suffering from a disease, in particular a myeloproliferative disease, or indications that such markers have been accomplished. Those in need of treatment include those diagnosed with the disorder, those suspected of having the disorder, those predisposed to have the disorder as well as those in whom the disorder is to be prevented. Hence, the mammal to be treated herein may have been diagnosed as having the disorder or may be predisposed or susceptible to the disorder.

The monolayers of the invention may be detected and/or imaged according to any methods known in the art and/or described herein. The particular imaging method is not critical and may be decided according to the knowledge of the person of skill in the art. In certain embodiments, the images obtained by the methods of the present invention comprise direct images of the cell. The imaging may or may not require the use of a dye or stain, may comprise imaging of both stained and non-stained components and/or may comprise imaging under conditions wherein the stain is or is not visible (e.g., imaging in bright-field (wherein a fluorescent stain would not be visible) and under UV-lighting (wherein a fluorescent stain would be visible), or combinations thereof). Imaging under bright-field conditions is well known and routine used in the art and may be performed according to standard methods and/or as described herein. Additionally, or alternatively, any other label-free imaging may be used in accordance with the invention. Such label-free methods are known and include, e.g., PhaseFocus imaging (Phase Focus Ltd, Sheffield, UK). In further embodiments, the invention relates to methods wherein the images obtained by the methods of the present invention provide images that enable the identification of cellular or subcellular structures, aspects or processes measuring about 0.25 μm in size or larger. In certain embodiments, said images may enable the identification of cellular or subcellular structures, aspects or processes measuring about 1.0 μm in size or larger.

The detection/imaging method may also be automated according to standard methods known in the art. For example, various computer-implemented methods exist that enable a person skilled in the art to analyze and interpret the microscopy images of cells obtained by the methods of the invention or to establish automated protocols for their analysis. For primary image analysis, including the correction for illumination bias in microscopy images, the identification of individual cells from microscopy images and the measurement of marker intensities and textures as well as nuclear and cellular size and shape parameters, the opensource software CellProfiler (e.g. version 2.1.1) can be used. Identification of marker-positive cells (such as CD34+progenitor cells or viability dye positive cells) can be performed by machine learning using the opensource software CellProfiler Analyst (e.g. version 2.0) and double-or triple-positive cells can be identified by a sequential gating strategy. Plate-overviews for further analysis and hit selection can be created using CellProfiler Analyst as well.

The cellHTS package in Bioconductor (e.g. version 2.14), or Pipeline Pilot (e.g. version 9.0; Accelrys), can both be used for the data analysis subsequent to the primary image analysis, including plate-effect normalization, control-based normalization, and hit selection.

Commercial automated microscopy systems may also be used in the practice of the invention, e.g., PerkinElmer Operetta automated microscope (PerkinElmer Technologies GmbH & Co. KG, Walluf, Germany), which systems may include corresponding image analysis software, e.g., PerkinElmer's Harmony software (e.g. version 3.1.1). Such automated and/or commercial systems can be used to perform primary image analysis, positive cell selection and hit selection from microscopic images according to the methods of the invention.

It is one advantage of the methods of the present invention that both healthy and diseased cells may be comprised in the monolayer. “Diseased cells” within the meaning of the invention relate to cells being affected by a disease and thus distinguishable from healthy cells. In particular, in some embodiments, a diseased cell will show differential expression of marker molecules or differential histological staining or differential morphologies that enable their specific detection using the methods of the present invention. For example, a diseased PBMC sample may show expression of known cancer markers, in particular markers for lymphoma or leukemia, which enable their detection and discrimination from healthy PBMCs. As another example, a diseased PBMC sample may show the presence of disease-associated cell morphologies revealed by histological staining, which enable their detection and discrimination from healthy PBMCs.

Diagnosis of the disease state of the subject may be made by standard methods routinely performed by those skilled in the art, e.g., physicians, but may also be supplemented or replaced with the methods of the present invention. For example, in order to determine whether a subject suffers or is likely to suffer from a disease, a cell-cell interaction pattern is determined that is characteristic for the respective disease using samples from subjects known to suffer from the disease. Additionally, or alternatively, the cell-cell interaction pattern of a healthy donor may be used to determine differences that likely are due to the respective disease. Cell interaction pattern here refers to the propensity of one or more different cell types or cell populations to interact with each other determined according to the present invention.

In this regard, the present invention can offer, in various embodiments, multiple advantages. In particular, samples are preferably treated shortly after isolation, which has several advantages: drug specificity and toxicity to, e.g., cancer cells is directly compared to that of the healthy cells from the patient, and complex aspects of drug responses can be measured that arise from the cell-cell interactions present in human blood. This ex-vivo analysis is a strong predictor of the clinical response of the patient, especially in complex genetic backgrounds, improving long-term therapeutic benefits. For example, drug responses of acute myeloid leukemia (AML) patients to single and combinatorial treatments of current and promising anti-cancer agents can be assessed. Furthermore, simultaneously measuring AML-specific biomarkers, such as the expression of CD34+ progenitor marker in the periphery is assessable by the methods of the invention. Furthermore, measurement of AML-specific cell morphologies and histological staining patterns is assessable by the methods of the invention. In addition, it is possible for future patients to have their drug responses characterized at several stages during the course of a disease. Accordingly, the present invention also provides, in some aspects, a PMBC monolayer for use in a diagnostic method for determining whether a subject suffering from or predisposed to a disease will respond or is responsive to treatment with a therapeutic agent.

In one embodiment, the invention relates to a method wherein said assessment in step (e) comprises applying a computer-implemented image analysis, such as an artificial neural network, for the analysis of the cell monolayer, the analysis comprising i) detecting cell locations by object detection; ii)
determining one or more cellular characteristic(s) in the cell monolayer; and iii) determining the response of the cells in the cell monolayer to the therapeutic agent, based on the one or more cellular characteristic(s) determined in step (ii).

In certain embodiments, the invention relates to methods wherein wherein the one or more cellular characteristic(s) comprise(s) cell viability, cell morphology, cell-cell interactions, membrane integrity, the identification or measurement of a biomarker and/or the presence of a pathogen or a combination thereof.

As described hereinabove, any computer-implemented image analysis may be used in the methods of the invention, however it is preferred that the computer-implemented image analysis is a machine learning analysis comprising, but not limited to, a weighted decision tree, a bootstrap aggregated decision tree, an artificial neural network, a linear discriminator, a non-linear discriminator or a combination thereof of any two or more machine learning analyses.

It is preferred that the computer-implemented image analysis method comprises elements that are able to detect cell locations by object detection. As used herein, the terms “detect”, “detecting” or “detection may describe either the general act of discovering or discerning or the specific observation of, inter alia, a cell, cellular characteristic or a molecule, whether labeled or not with a detectable label.

In some embodiments the machine learning analysis comprises an artificial neural network (ANN). An artificial neural network is a type of computational system capable of learning the relationship between an input data set and a target data set. An ANN is a simplified mathematical representation of a portion of the human neural system, intended to capture its “learning” and “generalization” abilities. ANNs are a major foundation in the field of artificial intelligence. ANNs are widely applied in research because they can model highly non-linear systems in which the relationship among the variables is unknown or very complex. ANNs are typically trained using a data set and a target. The data set is conventionally divided into a training set, a test set, and, in some cases, a validation set. A target is specified that contains the correct classification of each sample in the data set. In particular, a type of neural network called a feed-forward back-propagation classifier can be trained on an input data set to classify input samples as belonging to a pre-defined category according to a target. The category may, for example, refer to a disease, a disease state or a condition. A set of samples from multiple categories is repeatedly presented to the ANN classifier input, and for each sample presented during training, the output generated by the ANN is compared with the desired target. The difference between the target and the set of input samples is calculated, and the ANN is modified using the back-propagation algorithm to cause the output to more closely approximate the desired target value. After a large number of training iterations, the ANN output will closely match the desired target for each sample in the input training set.

Subsequently, when a new sample, not used during training, is presented to the ANN, it may generate an output classification value indicating which of the categories the new sample is most likely to fall into. The ANN is said to be able to “generalize” from its training to new, previously unseen input samples. This feature of ANNs allows them to be used to classify almost any input data which has a mathematically formulatable relationship to the category to which it can be assigned.

Whereas conventional software programs may require writing specific instructions to perform a function, ANNs may be programmed by training them with a known sample set and allowing them to modify themselves during training so as to provide a desired output such as a classification value. After training, when they are presented with new sample data, they can generalize what they have learned during training to be able to classify the new previously unseen data.

In some embodiments, the ANN of the present invention may be a convolution neural network (CNN). The architecture of a CNN is designed to take advantage of the 2D structure of an input image. This is achieved based on local connections with weights followed by a form of pooling which results in more efficient detection of translation invariant features. A benefit of CNNs is that they are easier to train and have fewer parameters than fully connected networks with the same number of hidden units. Each convolutional layer of a CNN is a set of triplets of convolution, non-linear, and pooling layers that enable the model to learn, extract and enhance implicit features of an image. The triplet layer as a whole is called the convolutional layer. When stacked together, the first layers act like a feature filter such as an edge enhancer and allow the convolutional layer to extract local features which are passed to deeper convolutional layers which act like increasingly more global feature extractors. A CNN comprises one or more convolutional layers (often with a subsampling step). Further, the one or more convolutional layers are followed by one or more fully connected layers as in a standard multilayer neural network. It is called a “Deep” CNN because it has multiple hidden convolutional layers. Each convolutional layer contains a set of feature maps, or filters, that extract features from a region of units using a convolution. Then an additive bias is applied and the result is passed through a sigmoid function. In a CNN the convolution layers are applied on 2D feature maps to compute spatial features.

The application of ANNs for object detection is well-understood in the art. Recent designs use deep CNNs to locate objects, for example, OverFeat trains a convolutional layer to predict box coordinates for multiple class-specific objects from an image pyramid. MultiBox generates region proposals from a network having an output layer that simultaneously predicts multiple boxes that are used for region-based CNN (R-CNN) object detection. YOLO also predicts bounding boxes and class probabilities directly from full images in one evaluation. All these methods use shared computation of convolutions, which has been attracting increased attention due to its relatively efficient and accurate visual recognition. Object detection often involves multi-task learning, such as landmark localization, pose estimation, and semantic segmentation.

In some embodiments, the methods of the present invention use YOLO for object detection. The term YOLO (an abbreviation of “You Only Look Once”) used herein refers to an image recognition architecture for detecting different classes of objects. In a YOLO system, a neural network is used to recognize and detect a full image. The neural network divides the image into multiple regions and predicts bounding boxes and probabilities for each region. These bounding boxes are weighted by the predicted probabilities. The detections can be threshold by some value to only see high scoring detections.

As stated hereinabove, in some embodiments a computer-implemented image analysis, preferably a machine learning analysis, preferably comprising an artificial neural network is used to determine the one or more cellular characteristic(s) in the cell monolayer. Cell characteristics may include, but are not limited to, cell morphology, cell-cell interactions, cell viability, membrane integrity, identification or measurement of a biomarker, nucleus morphology, cell motility, adhesion maturation and/or the presence of a pathogen or a combination thereof. Biomarkers comprise a broad range of biochemical entities, such as nucleic acids, proteins, lipids, carbohydrates, small metabolites, and cytogenetic and cytokinetic parameters.

In some embodiments, the methods of the invention not only provides the ability to identify and characterize cells and cellular characteristics, but also to determine the response of the cells and cell subpopulations to a therapeutic agent based on the cellular characteristics. Therefore, the methods of the invention are able to attribute cellular characteristics and/or changes in the cellular characteristics to adjust, confirm or nullify the effect of the therapeutic agent, thus enabling therapeutic decisions or therapeutic agent monitoring, i.e. drug monitoring. Furthermore, the methods of the invention, in some embodiments, may provide a prognosis based on the response of the cells and cell subpopulations to the therapeutic agent.

For the detection, identification and analysis of the cell and cellular characteristics, as well as the response of the cells and cell subpopulations to a therapeutic agent, it is preferred that a plurality of cellular characteristics are analyzed using the methods of the present invention. As the behavior and characteristics of both healthy and diseased cells, such as cancer cells, can be complicated, multiple cellular characteristics enable practitioners to capture the complex nature of the cell. Furthermore, a weighted significance may be attributed to two or more cellular characteristics such that their evaluation by the methods of the present invention, such as by a machine learning decision tree, carries either more or less significance in the overall determination of the pathological state or response to the therapeutic agent of the cell or cell sub-population.

In certain embodiments, the invention relates to methods wherein steps (a) to (e) are repeated for different concentrations of the therapeutic agent to determine an optimal concentration.

To determine whether a cell donor suffering from a disease will respond or is responsive to treatment with a therapeutic agent the methods of the invention may be repeated in order to determine the selectivity of the therapeutic agent. The methods in particular may be repeated for monolayers obtained from the same cell donor and cultured with the same therapeutic agent at different concentrations or may be repeated using a monolayer obtained from a single cell donor that has been divided into at least two parts and cultured with the same therapeutic agent at different concentrations. Determining the selectivity of a therapeutic agent at different concentrations of the therapeutic agent and/or determining whether a subject suffering from a disease will respond or is responsive to treatment with a therapeutic agent at different concentrations of the therapeutic agent may provide further improved results, because effective concentrations in vivo may vary depending on dosage and timing of an administered therapeutic agent. Thus, the methods of the invention, may, in some embodiments, determine the potential effects associated with the concentration of the therapeutic agent. The skilled person is aware of typical concentrations used in methods known in the art to determine selectivity of a test compound. That is, concentrations will typically be between 100 μM and 100 pM, preferably concentrations of 10 μM and 1 μm, more preferably of 10 μM, 1 μM and 100 nM are used.

In further embodiments, the invention relates to methods wherein the therapeutic agent is assigned a drug response score based on the response of the cells and/or cell population(s) as determined by the one or more cellular characteristic(s).

An important feature of the present invention is the focus on the analysis of cellular characteristics which are affected by a given therapeutic agent, therefore the affect, i.e., the change in an existing cellular characteristic or the appearance of a novel cellular characteristic, as a result of the therapeutic agent may provide useful information to predict the efficacy of a therapeutic agent. A comparison and/or matching may be computationally made using the methods of the invention between the cellular characteristics beneficially affected by the therapeutic agent in a monolayer identified to have a disease and a control sample identified to not have a disease, obtained from the same or different cell donor. Furthermore, in certain embodiments, the invention provides a method for identifying a number of cellular characteristics affected by the therapeutic agent, which may optionally be weighted and/or measured to provide quantitative data for computational analysis such that a drug response score may be generated. The drug response score, as understood herein, is a value provided by the methods of the invention to indicate whether the subject will be beneficially affected by the therapeutic agent thus reaching a healthy or improved disease condition.

A remarkable feature of this invention is that such modelling has prognostic value despite being to some degree a “blind” measure of the effect of a therapy in a subject. That is, the invention does not seek to identify cellular characteristics that are mechanistically or functionally linked to disease pathways or to the therapeutic mode of action. Rather, the invention looks only at the changes in cellular characteristics and its variation between samples, correlating this variation with the therapeutic endpoint. In the methods of the invention, any cellular characteristic that is influenced by the treatment and whose expression correlates with therapeutic endpoint is taken into account when producing the predictive model-it forms part of the panel of cell characteristics on which the logistic regression is conducted. Even without attempting to assign mechanistic or functional explanations to the cell characteristics involved, the invention can be used to identify a core set of cell characteristics of prognostic significance.

In specific embodiments, the invention relates to methods wherein the disease is a solid or liquid cancer and wherein the drug response score is determined by calculating the number of cancer cells determined in step (ii) divided by the average number of cancer cells in a control sample, divided by the number of non-cancer cells determined in step (ii), divided by the average number of non-cancer cells in a control sample.

As is shown in the appended Examples, the methods of the invention are of particular value to the analysis of cancer cell images. The inventors examined AML and GBM cell samples using the methods of the invention and were able to detect and/or identify healthy and cancerous cells and cell subpopulations using a computer-implemented image analysis method, in this case a ANN method. The ANN method then identified characteristic differences in cell characteristics, in particular cell morphology, such as aberrant nuclei, to identify malignant subpopulations and determine cellular linages. Once the cells and cell subpopulations were identified, drug response scores, i.e. the relative cytoxicity of cancer cells to selected drugs, were statistically determined and optimal drug regimens for the subjects predicted.

In certain embodiments, the invention relates to a method wherein subsequent to step (c), a step (d) comprising staining the monolayer. In a further embodiment, the inventions relates to methods wherein said staining involves staining with a histological stain. Moreover, In a specific embodiment, the invention relates to a method wherein the histological stain is a hematoxylin and eosin stain (H & E stain), hematoxylin stain, eosin stain, Giemsa stain, Pappenheim stain, May-Gruenwald stain, methylene blue stain, azure B stain, Masson triple or trichrome stain, aldehyde fuchsin stain, Verhoeff-van Gieson elastic tissue stain, silver stain, Periodic acid-Schiff stain, Gram stain, Papanicolau's (Pap) stain, or Immunoperoxidase stain, or a combination thereof.

In one embodiment, the invention relates to a method for selecting a therapeutic agent for use in the treatment of a disease of a subject having said disease, wherein said therapeutic agent is selected from at least two or more test agents, wherein each of the at least two or more test agents is tested in an assay comprising a) providing a cell population; b) culturing said cell population in a liquid medium comprising the test agent; c) air-drying the cell population with a sample evaporator to create a cell monolayer; d) staining the cell monolayer, preferably with a histological stain; e) imaging the cell monolayer created in step (d); and f) assessing the response of the cell monolayer by applying a computer-implemented image analysis for the image obtained in step (d), said analysis comprising i) detecting cell locations by object detection; ii) determining one or more cellular characteristic(s) in the cell monolayer; and iii) determining the response(s) of the cell population(s) in the cell monolayer to the test agent based on the one or more cellular characteristic(s) determined in step (ii), wherein the assay is repeated for each of the at least two or more test agents and wherein each of the test agents is assigned a drug response score based on the response(s) of the cell population(s) in the cell monolayer as determined by the one or more cellular characteristic(s) identified in step (ii). In a further embodiment, the invention relates to a method wherein the cellular characteristic comprises cell viability, cell-cell interactions, cell morphology, membrane integrity, the identification or measurement of a biomarker and/or the presence of a pathogen or a combination thereof.

One important advantage of the methods of the present invention is that the imaging, detection and analysis of the cell monolayer may be easily adapted to perform high-throughput and high-content screening of therapeutic agents, i.e. test agents or drugs. Accordingly, the invention also relates to a method for determining which of two or more or several test agents will most likely give the best clinical benefit to a patient suffering from a disease, such as cancer, whereby the methods of the invention are repeated for two or more test agent with the optimal drug response score is chosen.

In some embodiments, the invention relates to a method wherein the method is fully automated, that is, all the method steps described hereinabove are automated.

Since the methods of the present invention are particularly simplistic and robust, all method steps may be automated, in particular to provide a high-throughput high content screening platform for clinical settings. Any automated cell culture device known to those skilled in the art may be used in the methods of the invention, which typically features, inter alia automated liquid handling means, automated staining and washing means and automated means for dispensing therapeutic agents. Known devices may be adapted to feature the sample evaporator of the present invention. Moreover, automated microscopes and/or cameras may be used to capture images of the cell monolayer, which is connected to a computer-implemented image analysis means which may provide the analysis data on a screen to the user. Such an automated process substantially reduces manpower requirements and processing/analysis time which is particularly advantageous in clinical settings.

In some embodiments, the invention relates to the use of a monolayer created by the methods of the invention for determining whether a cell donor suffers from a disease. In a further embodiment, the invention relates to the use of a monolayer created by the methods of the invention for determining whether a subject suffering from a disease will respond or is responsive to treatment with a therapeutic agent. More specifically, in some embodiments, the invention relates to the use of said monolayer wherein the disease is a solid or liquid cancer, preferably wherein said solid or liquid cancer is carcinoma, sarcoma, myeloma, leukemia, lymphoma or a mixed-type cancer, more preferably wherein said solid or liquid cancer is glioblastoma (GBM) or acute myeloid leukemia (AML).

In one embodiment, the invention relates to a culture device comprising a monolayer created by the methods of the present invention wherein the culture device comprises a multi-well plate. Moreover, in a further embodiment, a culture device further comprising a sample evaporator. In specific embodiments, said sample evaporator comprises a plurality of air outlets located above and/or partially within the multi-well plate. In other embodiments, said multi-well plate comprises flat-bottomed wells.

The methods of the present invention are particularly enabled by the use of a culture device comprising a multi-well plate. Preferably the multi-well plate comprises several or all plate's wherein the wells form individual cell culture devices according to the invention. The multi-well plate may have, for example, 96 wells, 384 wells or 1536 wells. Thus, the multi-well plate provides the ability to separately maintain multiple cultures, e.g., for multiple perturbations, i.e., for multiple therapeutic agents or single therapeutic agents at multiple concentrations and with minimal material requirements, e.g., minimal media requirements.

The cell culture device may also further comprise a sample evaporator which enables the methods of the present invention. The sample evaporator preferably comprises a plurality of air outlets, preferably an air outlet is provided for each individual well of the multi-well plate. Therefore, the sample evaporator preferably comprises 96 outlets, 384 outlets or 1536 outlets. The size and shape of the air outlets are not particularly limited, however, it is preferred that the air outlet is a pipe or needle like structure. To facilitate the drying of the cell monolayer, it is preferred that the air outlets are arranged directly above the multi-well plate, wherein each air outlet is arranged directly and centrally above well.

The bottom wall and/or an inner bottom surface of the cell culture device may be substantially flat and/or horizontal, i.e., forming flat-bottomed wells. This may be beneficial for achieving a cell monolayer during incubation and preventing cell agglomerations. Optionally, the inner bottom surface of the device may be coated and/or treated so as to promote cell adhesion. For example, the inner bottom surface of the device may be coated with molecules or compounds that promote cell adhesion. Non-limiting examples of known agents that promote cell adhesion include, but are not limited to, polylysine, fibronectin or gelatin. The invention, however, does not exclude the use of devices that have not been treated and/or coated so as to promote cell adhesion. It may be preferable that the inner bottom surface of the device is not coated and/or microstructured.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

The term “subject”, as used herein, often refers to an aminal, including, but not limited to, a primate (e.g., human). The terms “subject”, “patient” and “cell donor” are used interchangeably herein.

The term “sample”, as used herein, refers to any substance containing or presumed to contain a cell of interest or a cell for investigation in the methods of the invention. The term “sample” thus includes a cell, organism, tissue, fluid or substance including but not limited to, for example, blood, plasma, serum, spinal fluid, lymph fluid, synovial fluid, urine, tears, stool, external secretion of the skin, intestinal or genitourinary tracts, blood cells, tumors, organs, tissue, samples of cell culture constituents, cell lines, plant cells, natural isolates (such as drinking water, seawater, solid materials), microbial specimens and processed, purified, isolated, enriched or enhanced derivatives thereof. The sample may require preliminary processing designed to, purify, isolate, or enrich the sample for cells of interest. A variety of techniques known to those skilled in the art may be used for this purpose.

The phrase “machine learning”, as used herein, refers to the construction and adapting of algorithms based on data with minimal instructions. See, e.g., C. M. Bishop, Pattern Recognition and Machine Learning (Springer 2007).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### EXAMPLES

### Experimental Models

The invention was developed and optimized with primary immune cells from buffy coat samples which were obtained from coded healthy donors provided by the Blutspende Zurich, under a study protocol approved by the cantonal ethical committee Zurich (KEK Zurich, BASEC-Nr 2019-01579). Results presented also include samples collected from patients with newly diagnosed acute myeloid leukemias undergoing intensive induction chemotherapy. The recruitment of patients occurred through the project leader or the team of treating physicians of the Hematology Division of the University Hospital Zurich (USZ). The research project was carried out in accordance with the research plan and with principles enunciated in the current version of the Declaration of Helsinki (DoH), the Principles of Good Clinical Practice (GCP), the Swiss Law and Swiss regulatory authority's requirements as applicable. Ethical approval was granted by KEK Zurich, (BASEC-Nr: 2018-01547). The results showcase the use of solid tumors in the form of primary patients derived glioblastoma cells lines collected under the Ethical approval of the KEK Zurich.

### Example 1

### Single Cell Collection, Purification and Drug Screening

To isolate cells from bone marrow aspirate or peripheral blood, samples were diluted in PBS (Gibco)+2 mM EDTA (Sigma-Aldrich) and purified using a Lymphoprep density gradient (STEMCELL Technologies) according to the manufacturer's instructions. The resulting cells at the interface were collected, washed once with PBS+EDTA and resuspended in RPMI 1640+GlutaMax medium (Gibco) supplemented with 10 % human serum (Chemie Brunschwig). For glioblastoma derived cell lines, cells were obtained from culture and cell number and viability was determined by use of a Countess II Cell Counter (Thermo Fisher). The single-cell suspension of immune cells were seeded (2*104 cell/well for immune cells or AML samples and 2*103 for glioblastoma cells with 50 pl/well) in CellCarrier 384 Ultra, clear-bottom, tissue-culture-treated plates (PerkinElmer) containing for example antineoplastic agents, immunostimuli, immunotherapeutics, bioactive compounds or approved therapies and incubated overnight (24 h at 37 °C and 5 % CO₂).

### Example 2

### Monolayer Creation

After a few hours in growth media, all cells were either adherent to the imaging plate or sunk to the bottom of the plate. After 24 h of incubation alone or with biological or chemical compounds, including antineoplastic agents, the majority of the growth media was subsequently removed by use of a HydroSpeed plate washer (Tecan). The plate washer was set to not disturb the bottom of the plate and 10 ul of growth media with the undisturbed cellular layer remained in the imaging plate. This step is not obligatory but helps to reduce the time of subsequent air-drying of the plate. After removal of the majority of growth media, cells were fully air-dried with a 384 sample evaporator with non-heated pressurized air at 1.5 L/min/mm² for 1 h. This process immobilizes all cells and fixes them to the surface of the multi-well screening plate.

### Example 3

### Pappenheim Staining of Aml Samples and Image Acquisition

After air-drying, cellular samples were fixed and stained with hematological stains. For all AML results presented in this proposal, samples were stained with 20 ul/well filtered May-Gruenwald stain (Sigma) for 5 min. Subsequently, samples were washed once with a plate washer and phosphate buffered solution (Sigma, pH of 6.8) and further stained with 20 ul of 1:10 diluted giemsa stain (Sigma, pH of 6.8 phosphate buffer) for 15 min. The stained wells were washed twice with phosphate buffer solution and air-dried for storage. The multi-well plates were then stored in the dark until they were used for image acquisition. Images were acquired with standard automated microscopy set to a color bright-field mode.

### Example 4

### Cell Detection/Image Analysis

For the purposes of detecting cells in the acquired color images, deep learning based object detection was used, namely YOLOv2 and YOLOv3 framework (YOLO9000: Better, Faster, Stronger, Joseph Redmon, A N Farhadi; 2017 & YOLOv 3: An Incremental Improvement, Joseph Redmon, AN Farhadi; 2018). Cellular identity was determined either directly using the classification output of the YOLO network, or indirectly by deploying custom deep convolutional neural networks (CNN) with an adapted ‘Alex-Net’ architecture (A. Krizhevsky, I. Sutskever. and G. E. Hinton, Advances in Neural Information Processing Systems 25, 2012). Hand curated datasets were used in training, validation and testing of the object detection and CNN frameworks. Neural networks used in this work were implemented using MATLAB's Neural Network Toolbox Version R2020a.

### Example 5

### Drug Response Analysis

Drug response scores were calculated as the number of cells of population X (identified by the CNN) divided by the average number of cells of population X in a control sample, divided by the number of other cells divided by the average number of other cells in a control sample. Antibody-based treatments were normalized towards their respective isotype control, whereas all other drugs were normalized towards dimethyl sulfoxide (DMSO). For calculation of ex-vivo drug responses, all drug score values per sample were averaged over technical replicates, and subsequently zero-centered (1-drug response score). Thus, a positive score represents a relative reduction of that population (on-target effect), whereas a negative score indicates relative ex vivo chemoresistance.

### Example 6

### Direct Detection of a Pathogen

A peripheral blood sample is isolated from a patient suspected of Plasmodium infection. Similar or the same methods as examples 1 to 5 are used for the preparation and imaging of the cell monolayer(s). The cell monolayer is typically stained with a Giemsa-stain for diagnosis, to assess degree of infection and to identify the specific Plasmodium species. Giemsa stain highlights the parasite in a distinct manner and is also used to differentiate the nuclear and cytoplasmic morphology of platelets, red blood cells, white blood cells. Parasites, white blood cells and platelets, are a bluish purple color when stained. Red blood cells are usually colored in slight pink. The rings of the trophozoites take up pale blue color. The parasitemia is measured by counting the number of parasites present in the specimen. Morphological assessment of, for example, granulometry, shape and texture of the red blood cells is used to determine the specific Plasmodium species. Drug screening is performed on the cell monolayer(s), allowing for the identification of new drug candidates.

### Example 7

### Indirect Detection of a Pathogen

A peripheral blood sample is isolated from a patient suspected of viral infection. Similar or the same methods as examples 1 to 5 are used for the preparation and imaging of the cell monolayer(s). Cytopathic effects (CPE)-structural changes in the host cell(s), are examined and analyzed for the diagnosis and characterization of infection. Cytopathic effects include rounding of the infected cell, vacuolization, swelling/clumping of host cell, fusion with adjacent cells to form syncytia and the appearance of nuclear or cytoplasmic inclusion bodies, which are accumulations of virus replication by-products, altered host cell organelles and/or structures. CPE is used for diagnosis and drug screening, wherein a reduction in CPE acts as an indicator for the efficacy of a test compound/drug.

### Example 7

### Wright-Hiemsa Staining of Whole Blood Samples

After air-drying, cellular samples obtained from whole blood samples were fixed and stained with hematological stains. Air-dried samples were fixed with ice-cold 50 ul/well pure Methanol for 10 min. Subsequently, samples were washed once with a plate washer and phosphate buffered solution (Sigma, pH of 6.8) and further stained with 50 ul of 1:10 diluted wright-giemsa stain (pH of 6.8 phosphate buffer) for 15 min. The stained wells were washed once with phosphate buffer solution, twice with water and air-dried for storage. The multi-well plates were then stored in the dark until they were used for image acquisition. Images were acquired with standard automated microscopy set to a color bright-field mode.

### Example 8

### Fluorescent Staining of Air Dried Samples

After air-drying, cellular PBMC samples were fixed and stained with fluorescent antibodies. Here samples were fixed with 20 ul/well 6% PFA (Sigma) for 10 min. Subsequently, samples were air-dried for monolayer creation washed once with a plate washer and phosphate buffered solution (Sigma, pH of 7.4) and further stained with 20 ul of 1:300 diluted antibody mix containing fluorescent antibodies targeting the T-cell surface proteins CD 4, CD 8 and CD 3 (Biolegend) and the nuclear dye DAPI (1:5000) for 1 hour. The stained wells were washed once with phosphate buffer solution. The multi-well plates were then stored in the dark until they were used for image acquisition. Images were acquired with an Opera Phenix High Content Screening System.

Example 9

### Combined Hematological and Fluorescent Staining of Air Dried Samples

After air-drying, cellular PBMC samples were fixed and stained with hematological stains. Air-dried samples were fixed with ice-cold 50 ul/well pure Methanol for 10 min. Subsequently, samples were washed once with a plate washer and phosphate buffered solution (Sigma, pH of 6.8) and further stained with 50 ul of 1:10 diluted wright-giemsa stain (pH of 6.8 phosphate buffer) for 15 min. The stained wells were washed once with phosphate buffer solution, twice with water. Samples were subsequently stained with 1:2500 diluted DAPI (in PBS) for 15 min, washed once and air-dried for storage. The multi-well plates were then stored in the dark until they were used for image acquisition. Images were acquired with standard automated microscopy set to a color bright-field mode and subsequently with an Opera Phenix High Content Screening System.

1. A method for the creation of a cell monolayer comprising
a) providing a cell population;
b) culturing said cell population in a liquid medium; and
c) air-drying the cell population with a sample evaporator to create a cell monolayer.

2. The method of claim 1, wherein naturally occurring cell-cell interactions, cell/membrane integrity are maintained during the formation of the monolayer.

3. The method of claim 1, wherein the cell population is incubated at a density of about 100 cells per mm² to about 30000 cells per mm²; and/or wherein the cell population is obtained from a primary cell culture, a cell line or a cell strain, a primary patient sample, a cell donor or a combination thereof; and/or wherein the cells are cultured in a multi-well plate.

4-5. (canceled)

6. The method of claim 1, wherein air-drying immobilizes the cells onto a surface; and/or wherein the cell population comprises non-adherent cells or a mixture of non-adherent and adherent cells.

7. (canceled)

8. The method of claim 1, wherein the sample evaporator supplies pressurized air at an air speed of 0.5 to 2.5 liters per minute per mm² (L/min/mm²). .

9. The method of claim 1, wherein (i) air-drying is continued until the cell population is dry and the cell monolayer is created, (ii) the cell population is air-dried between 10 min and 12 hrs, or (iii) the temperature of the supplied air is between 10 to 50 °C.

10. (canceled)

11. The method of claim 1, wherein prior to air-drying the cell population, part of the liquid media is removed, wherein the liquid media is removed until at least 1 mm of liquid media remains above the surface of the cell population.

12. (canceled)

13. The method of claim 1, wherein (i) the method further comprises a step of chemically fixing the cell monolayer; and/or (ii) the method further comprises, subsequent to step (c), a step (d) comprising staining the monolayer with a histological stain, a fluorescent stain, or both a histological stain and a fluorescent stain.

14-18. (canceled)

19. A method for determining whether a cell donor suffers from a disease, the method comprising
a) providing the cell monolayer created by the methods of claim 3;
b) analyzing the cell monolayer; and
c) determining whether the cell donor suffers from a disease.

20. The method of claim 19, wherein the disease is an infectious disease, solid cancer or liquid cancer.

21-23. (canceled)

24. A method for determining whether a cell donor suffering from a disease will respond or is responsive to treatment with a therapeutic agent comprising
a) providing a cell population;
b) culturing said cell population in a liquid medium comprising the therapeutic agent;
c) air-drying the cell population with a sample evaporator to create a cell monolayer;
d) imaging the cell monolayer created in step (c); and
e) assessing the response of the cells in the cell monolayer to the therapeutic agent to determine whether the cell donor suffering from a disease will respond or is responsive to treatment with the therapeutic agent.

25. The method of claim 24, wherein said assessment in step (e) comprises applying a computer-implemented image analysis, such as an artificial neural network, for the analysis of the cell monolayer, the analysis comprising
i) detecting cell locations by object detection;
ii) determining one or more cellular characteristic(s) in the cell monolayer; and
iii) determining the response of the cells in the cell monolayer to the therapeutic agent, based on the one or more cellular characteristic(s) determined in step (ii).

26. The method of claim 24, wherein steps (a) to (e) are repeated for different concentrations of the therapeutic agent to determine an optimal concentration.

27. The method of claim 24, wherein (i) the one or more cellular characteristic(s) comprise(s) cell viability, cell morphology, cell-cell interactions, membrane integrity, the identification or measurement of a biomarker and/or the presence of a pathogen or a combination thereof; and/or (ii) the therapeutic agent is assigned a drug response score based on the response of the cells and/or cell population(s) as determined by the one or more cellular characteristic(s).

28. (canceled)

29. The method of claim claim 27, wherein the disease is a solid or liquid cancer and wherein the drug response score is determined by calculating the number of cancer cells determined in step (ii) divided by the average number of cancer cells in a control sample, divided by the number of non-cancer cells determined in step (ii), divided by the average number of non-cancer cells in a control sample.

30. The method of claim 29, wherein the method further comprises staining the monolayer subsequent to air-drying the monolayer with the sample evaporator in step (c).

31. The method of claim claim 30, wherein said staining involves staining with a histological stain, a fluorescent stain, or both a histological stain and a fluorescent stain.

32-34. (canceled)

35. A method for selecting a therapeutic agent for use in the treatment of a disease of a subject having said disease, wherein said therapeutic agent is selected from at least two or more test agents, wherein each of the at least two or more test agents is tested in an assay comprising
a) providing a cell population;
b) culturing said cell population in a liquid medium comprising the test agent;
c) air-drying the cell population with a sample evaporator to create a cell monolayer;
d) staining the cell monolayer, preferably with a histological stain;
e) imaging the cell monolayer created in step (d); and
f) assessing the response of the cell monolayer by applying a computer-implemented image analysis for the image obtained in step (d), said analysis comprising
i) detecting cell locations by object detection;
ii) determining one or more cellular characteristic(s) in the cell monolayer; and
iii) determining the response(s) of the cell population(s) in the cell monolayer to the test agent based on the one or more cellular characteristic(s) determined in step (ii),
wherein the assay is repeated for each of the at least two or more test agents and wherein each of the test agents is assigned a drug response score based on the response(s) of the cell population(s) in the cell monolayer as determined by the one or more cellular characteristic(s) identified in step (ii).

36. The method of claim 35, wherein the cellular characteristic comprises cell viability, cell-cell interactions, cell morphology, membrane integrity, the identification or measurement of a biomarker and/or the presence of a pathogen or a combination thereof.

37-42. (canceled)

43. A culture device comprising a monolayer created by the methods of claim 1, wherein the culture device comprises a multi-well plate.

44-46. (Canceled)
